(19) United States
(12) Patent Application Publication
(10) Pub. No.: US 2026/0364227 A1
(43) Pub. Date: Nov. 19, 2026

(54) AQUATIC-DERIVED HYDROXYAPATITE BIOMATERIALS AND RELATED METHODS

(71) Applicants: Francisco Hernán MUÑOZ THOMSON, Las Condes, Santiago (CL); Salmoss SpA, Las Condes, Santiago (CL)

(72) Inventor: Francisco Hernán Muñoz Thomson, Las Condes, Santiago (CL)

(73) Assignee: Salmoss SpA, Las Condes, Santiago (CL)

(21) Appl. No.: 19/490,543

(22) PCT Filed: Sep. 25, 2024
(86) PCT No.: PCT/IB2024/000525

§ 371 Date: Dec. 5, 2025

(30) Foreign Application Priority Data
Sep. 29, 2023 (CL) 202302931

(51) Int. Cl. A61L 27/36; A61L 27/04; A61L 27/12; A61L 27/22; A61L 27/24

(52) U.S. Cl. CPC A61L 27/3608; A61L 27/047; A61L 27/12; A61L 27/222; A61L 27/225; A61L 27/24; A61L 2430/02

(57) ABSTRACT

The present invention relates to the obtaining of a non-cytotoxic biomaterial comprising hydroxyapatite obtained from aquatic-derived bone and promoting osteogenesis, which comprises hydroxyapatite obtained, D-tricalcium phosphate, calcium oxide, magnesium oxide, and/or amorphous biomaterial. The invention also generally relates to a composition including said biomaterial, and further comprising collagen, and to the use of said composition for the manufacture of a product useful for promoting osteogenesis.

### FIELD

The present invention generally relates to aquatic-derived biomaterials comprising hydroxyapatite or derivatives thereof, and related methods. For example, the present invention relates to methods for obtaining a biomaterial based on collagenized hydroxyapatite (HAPCS) from waste generated by the salmon industry.

### BACKGROUND

Osteogenesis techniques pose a significant challenge in the industry, leading to continuous development of biomaterials that promote this biological phenomenon. Particularly effective biomaterials in this regard are those based on hydroxyapatite, which finds applications in regenerative medicine, specifically in bone regeneration and tissue engineering. Currently available solutions for generating hydroxyapatite biomaterials typically come from human, bovine, equine, porcine, and synthetic origins.

However, there are ethical, religious, or cultural considerations that preclude the use of human, porcine, and bovine grafts in many countries worldwide, such as Kosher and Halal communities. Additionally, diseases like mad cow disease contraindicate the use of bovine-derived biomaterials. While synthetic derivatives lack the ethical and cultural considerations mentioned above, they offer lower clinical effectiveness, making them a less desirable option. In this context, there's a need to find alternative natural sources of biomaterials that are effective in osteogenesis, non-cytotoxic, and cost-effective.

Document IN202031021533 describes a hydroxyapatite biomaterial derived from the bones of the fish Puntius conchonius, poly(lactic-co-glycolic acid), and poly(methyl methacrylate), and a method for obtaining hydroxyapatite from P. conchonius bones. While this biomaterial provides an alternative to conventional sources, it requires combinations with additional polymers, complicating and increasing the cost of the process. Additionally, the resulting biomaterial demonstrated compressive strengths lower than 12 MPa, indicating a disadvantage due to its fragility.

Currently, there are no alternative hydroxyapatite-based biomaterials that are easily obtainable, low-cost, possess high mechanical strength, non-cytotoxic, and highly effective for osteogenesis. Accordingly, improved compositions, materials, and methods are needed.

### SUMMARY

The present invention generally relates to aquatic-derived hydroxyapatite biomaterials and related methods.

In one aspect, biomaterials are provided. In some embodiments, the biomaterial is an aquatic-derived biomaterial. In some embodiments, the aquatic-derived biomaterial comprises a first composition present in the biomaterial in an amount greater than or equal to 10 wt % and less than or equal to 100 wt % versus the total biomaterial weight, a second composition present in the biomaterial in an amount greater than or equal to 0.1 wt % and less than or equal to 90 wt % versus the total biomaterial weight, the first composition comprising greater than or equal to 50 wt % and less than or equal to 90 wt % an aquatic-derived hydroxyapatite or derivative thereof, optionally substituted, greater than or equal to 5 wt % and less than or equal to 40 wt % amorphous biomaterial, the amorphous biomaterial comprising: greater than or equal to 10 wt % and less than 20 wt % β-tricalcium phosphate versus the total weight of the first composition, greater than or equal to 0.1 wt % and less than or equal to 5 wt % calcium oxide, greater than or equal to 0.1 wt % and less than or equal to 20 wt % magnesium oxide, and the second composition comprising one or more of collagen and/or derivatives thereof, synthetic collagen and/or derivatives thereof, gelatin and/or derivatives thereof, fibrin and/or derivatives thereof, fibrin and/or derivatives thereof.

In another aspect, methods of producing aquatic-derived biomaterials is provided. In some embodiments, the method comprises removing non-bone material comprising incubating an aquatic-derived bone for greater than or equal to 20 minutes to less than or equal to 4 hours at a temperature of greater than or equal to 700 °C and less than or equal to 120 °C thereby removing non-bone material, deproteinizing the aquatic-derived bone comprising incubating the aquatic-derived bone in sodium hydroxide at a temperature of greater than or equal to 15 °C and less than or equal to 25 °C for greater than or equal to 30 minutes to less than or equal to 4 hours, thereby deproteinizing the bone, delipidating the aquatic-derived bone comprising incubating the aquatic-derived bone in acetone at a temperature of greater than or equal to 15 °C and less than or equal to 30 °C for greater than or equal to 4 hours to less than or equal to 8 hours, thereby delipidating the bone, drying the aquatic-derived bone at a temperature of greater than or equal to 50 °C and less than or equal to 70 °C for greater than or equal to 6 hours and less than or equal to 48 hours, grinding the dried bone, heating the ground bone to a temperature of greater than or equal to 800 °C and less than or equal to 1200 °C for greater than or equal to 1 hour and less than or equal to 3 hours thereby producing a aquatic-derived biomaterial, and adding a second composition comprising one or more of collagen and/or derivatives thereof, actin and/or derivatives thereof, gelatin and/or derivatives thereof, porogenic additives, and bone morphogenetic proteins to the aquatic-derived biomaterial.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows an exemplary method for producing an aquatic-derived biomaterial, according to one set of embodiments.

FIG. 2A shows the Raman spectra of exemplary hydroxyapatite biomaterials obtained during the optimization of the deproteinization stage, according to one set of embodiments. Curve 1 corresponds to the use of variables NaOH 2 M and 20 °C, curve 2 corresponds to the use of variables NaOH 2 M and 50 °C, and curve 3 corresponds to the use of variables NaOH 2.5 M and 20 °C.

FIG. 2B shows the Raman spectra of exemplary hydroxyapatite biomaterials obtained during the optimization of the delipidation stage, according to one set of embodiments. Curve 1 corresponds to the use of variables acetone and 6 hours, curve 2 corresponds to the use of variables acetone and 12 hours, curve 3 corresponds to the use of variables hexane and 6 hours, and curve 4 corresponds to the use of variables hexane and 12 hours.

FIG. 3 shows the Raman spectra of exemplary hydroxyapatite biomaterials obtained during the optimization of the calcination stage, according to one set of embodiments. Curve 1 corresponds to the use of variables 800 °C and 6 hours, curve 2 corresponds to the use of variables 800 °C and 4 hours, and curve 3 corresponds to the use of variables 800 °C and 2 hours.

FIG. 4 shows the Raman spectra of exemplary hydroxyapatite biomaterials obtained during the optimization of the calcination stage, according to one set of embodiments. Curve 4 corresponds to the use of variables 900 °C and 6 hours, curve 5 corresponds to the use of variables 900 °C and 4 hours, and curve 6 corresponds to the use of variables 900 °C and 2 hours.

FIG. 5 shows the Raman spectra of exemplary hydroxyapatite biomaterials obtained during the optimization of the calcination stage, according to one set of embodiments. Curve 7 corresponds to the use of variables 1000 °C and 6 hours, curve 8 corresponds to the use of variables 1000 °C and 4 hours, and curve 9 corresponds to the use of variables 1000 °C and 2 hours.

FIG. 6 shows the Raman spectrum of the exemplary hydroxyapatite biomaterial obtained by exemplary protocol 1, according to one set of embodiments.

FIG. 7 shows the Raman spectrum of the exemplary hydroxyapatite biomaterial obtained by exemplary protocol 2, according to one set of embodiments.

FIG. 8 shows the Raman spectrum of the exemplary hydroxyapatite biomaterial obtained by exemplary protocol 3, according to one set of embodiments.

FIG. 9 shows the X-ray diffractogram of the exemplary hydroxyapatite biomaterial obtained by exemplary protocol 1, according to one set of embodiments.

FIG. 10 shows the X-ray diffractogram of the exemplary hydroxyapatite biomaterial obtained by exemplary protocol 2, according to one set of embodiments.

FIG. 11 shows the X-ray diffractogram of the exemplary hydroxyapatite biomaterial obtained by exemplary protocol 3, according to one set of embodiments.

FIG. 12 shows the weight change as a function of temperature after performing thermogravimetric analysis on the exemplary hydroxyapatite biomaterial obtained by exemplary protocol 1, according to one set of embodiments.

FIG. 13 shows the weight change as a function of temperature after performing thermogravimetric analysis on the exemplary hydroxyapatite biomaterial obtained by exemplary protocol 2, according to one set of embodiments.

FIG. 14 shows the weight change as a function of temperature after performing thermogravimetric analysis on the exemplary hydroxyapatite biomaterial obtained by exemplary protocol 3, according to one set of embodiments.

FIG. 15 shows scanning electron microscopy images of the exemplary hydroxyapatite biomaterial obtained by exemplary protocol 1 at magnifications of 220× (left) and 4600× (right), according to one set of embodiments.

FIG. 16 shows scanning electron microscopy images of the exemplary hydroxyapatite biomaterial obtained by exemplary protocol 2 at magnifications of 1000× (left) and 2900× (right), according to one set of embodiments.

FIG. 17 shows scanning electron microscopy images of the exemplary hydroxyapatite biomaterial obtained by exemplary protocol 3 at magnifications of 1000× (left) and 2500× (right), according to one set of embodiments.

FIG. 18 shows the compression strength results, showing the force-distance curves for the hydroxyapatite biomaterials obtained by exemplary protocols 1, 2, and 3, according to one set of embodiments.

FIG. 19 shows the results obtained for cell viability and proliferation from MTT assays, according to one set of embodiments. Results at 24 hours are shown for cells cultured in the presence of commercial hydroxyapatite biomaterials of human origin, bovine origin, salmon origin from each of the 3 selected exemplary protocols, and a negative control.

FIG. 20 shows the results obtained for cell viability and proliferation from MTT assays, according to one set of embodiments. Results at 48 hours are shown for cells cultured in the presence of commercial hydroxyapatite biomaterials of human origin, bovine origin, salmon origin from each of the 3 selected exemplary protocols, and a negative control.

FIG. 21 shows 10× images of mesenchymal cells cultured for 21 days in the presence of exemplary hydroxyapatite biomaterials from exemplary protocol 1 (image 1), exemplary protocol 2 (image 2), exemplary protocol 3 (image 3), human allograft (image 4), bovine xenograft (image 5), hydroxyapatite biomaterial-free control (image 6), and undifferentiated cells (image 7), according to one set of embodiments.

FIG. 22 shows the size distribution of exemplary hydroxyapatite biomaterials obtained by exemplary protocols 1, 2, and 3, using a milling time of 2 seconds, according to one set of embodiments.

FIG. 23 shows the size distribution of exemplary hydroxyapatite biomaterials obtained by exemplary protocols 1, 2, and 3, using a milling time of 6 seconds, according to one set of embodiments.

FIG. 24 shows the size distribution of exemplary hydroxyapatite biomaterials obtained by exemplary protocols 1, 2, and 3, using a milling time of 10 seconds, according to one set of embodiments.

FIG. 25 shows images by polarized light microscopy of the materials obtained with milling times of 2 seconds, according to one set of embodiments. The upper left corresponds to exemplary protocol 1, upper right to exemplary protocol 2, and lower image to exemplary protocol 3.

FIG. 26 shows images by polarized light microscopy of the materials obtained with milling times of 10 seconds, according to one set of embodiments. The upper left corresponds to exemplary protocol 1, upper right to exemplary protocol 2, and lower image to exemplary protocol 3.

### DETAILED DESCRIPTION

The present invention generally relates to aquatic-derived biomaterials comprising hydroxyapatite or derivatives thereof, and related methods. In some embodiments, the aquatic-derived biomaterials comprise a mixture of a first composition comprising aquatic-derived hydroxyapatite or derivatives thereof and a second composition comprising collagen (or other scaffold proteins). Advantageously, the aquatic-derived biomaterials may comprise hydroxapetite and/or collagen (or other scaffold proteins) derived primarily from aquatic sources. For example, in some embodiments, the present invention relates to methods for obtaining a biomaterial based on collagenized hydroxyapatite (HAPCS) from waste generated by the fishing industry, such as the salmon industry. In some embodiments, the biomaterials may advantageously be substantially free of synthetic polymers.

In some embodiments, the invention generally relates to the obtaining of a biomaterial comprising hydroxyapatite, which is low-cost, non-cytotoxic, and/or promotes osteogenesis, from aquatically sourced bones, such as salmon bones, thus serving as an effective alternative to biomaterials derived from human or other natural sources. In some embodiments, the invention generally relates to a composition that includes said hydroxyapatite derived from the aquatically sourced bone, and additionally includes collagen and/or other scaffolding proteins (actin, gelatin, fibrin, amongst others). The biomaterials described herein may be useful in a number of applications including medical applications for treatment of a subject.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig.

Generally, the invention is directed toward use with humans. In some embodiments, a subject may demonstrate health benefits, e.g., upon administration of the aquatic-derived biomaterial. For example, the aquatic-derived biomaterials described herein may be useful for use as a xenograft (e.g., a xenograft comprising the aquatic-derived biomaterial comprising a first composition and a second composition). The term “xenograft” generally refers to a type of transplant that is performed between two different species. This process involves the transfer of tissues, organs, or cells from one organism (the donor) to another organism of a different species (the recipient). A common example of a xenograft is the use of pig tissue in repairing human heart valves. This technique is used in medical research and clinical medicine to study diseases, test treatments, and in some cases, directly treat conditions in patients.

In some embodiments, the aquatic-derived biomaterial described herein may be administered to a subject (e.g., for wound healing, tissue regeneration, amongst others). In some embodiments, the biomaterial is administered surgically (e.g., implanted). In some embodiments, the biomaterial is administered endoscopically. In some embodiments, the biomaterial is administered topically (e.g., on a skin and/or mucosal layer). Other administration routes are also possible (e.g., oral, ureteral, rectal, amongst others).

In some embodiments, the aquatic-derived biomaterial comprises a first composition (e.g., comprising an aquatic-derived hydroxyapatite or derivative thereof) and a second composition (e.g., comprising a protein scaffold such as collagen). The term “aquatic-derived” as used herein generally refers to a material derived from an aquatic source. For example, an aquatic-derived bone, as used herein, generally refers to bone found in an aquatic species. Non-limiting examples of aquatic source for the biomaterials described herein include fish (e.g., salmon, tuna, anchovy, angelfish, catfish, cod, sole, pike, char, herring, shark, kill fish, blowfish, bluefish, trout, eel, stingray, puffer fish, flounder, suckerfish, sturgeon, tigerfish, grouper, etc.), marine mammals (e.g., cetaceans (e.g., whale, dolphin, porpoise), pinnipeds (e.g., seal, sea lion, walrus), sirenians (e.g., manatees, dugongs), sea otters, etc.), crustaceans (e.g., shrimp, prawn, crab, lobster, crayfish, krill, barnacles, or the like) marine reptiles (e.g., iguanas, snakes, turtles, crocodiles), marine organisms (e.g., algae (e.g., seaweed, green algae, red algae), lichen, coral, sea sponge), amongst others.

In some embodiments, the aquatic-derived biomaterial is free of non-aquatic derived hydroxyapatite. For example, the aquatic-derived biomaterial does not comprise hydroxyapatite derived from bovine, human, equine, chicken, and/or swine. In some embodiments, the second composition is free of non-aquatic derived scaffold material. For example, in some embodiments, the scaffold material is not derived from bovine, human, equine, chicken, and/or swine. In some embodiments, the first composition is not derived from bovine, human, equine, chicken, synthetic or swine sources and the scaffold material is derived from a bovine, human, equine, chicken, synthetic, and/or swine source.

In some embodiments, the first composition comprises an aquatic-derived hydroxyapatite and/or derivatives thereof. In some embodiments, the aquatic-derived hydroxyapatite or derivative thereof is derived from the bone of a fish (e.g., salmon). For example, in some embodiments, the bone (e.g., from which the hydroxyapatite is derived) is obtained from a freshwater fish. In some embodiments, the bone (e.g., from which the hydroxyapatite is derived) is obtained from a saltwater fish. In some embodiments, the first composition is derived from a different organism than the second composition. In some embodiments, the first composition is derived from the same organism as the second composition. For example, in an illustrative embodiment, the first composition comprises hydroxyapatite and/or derivatives thereof derived from salmon bone, and the second composition comprises collagen derived from salmon skin. Those of ordinary skill in the art would understand based upon the teachings of this specification that other aquatic sources for the first composition and or the second composition are also possible.

The aquatic-derived biomaterials described herein may advantageously provide a biomaterial useful for xenografts which does not present ethical, religious, or cultural considerations (e.g., that preclude the use of human, porcine, and bovine grafts in many countries worldwide, such as Kosher and Halal communities) while remaining clinically effective and/or cost effective.

In some embodiments, the first composition may be present in the aquatic-derived biomaterial in any suitable amount. For example, in some embodiments, the first composition is present in the aquatic-derived biomaterial in an amount greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, greater than or equal to 90 wt %, greater than or equal to 95 wt %, or greater than or equal to 99.9 wt % versus the total weight of the aquatic-derived biomaterial. In some embodiments, the first composition is present in the aquatic-derived biomaterial in an amount less than 100 wt %, less than or equal to 99.9 wt %, less than or equal to 95 wt %, less than or equal to 90 wt %, less than or equal to 80 wt %, less than or equal to 70 wt %, less than or equal to 60 wt %, less than or equal to 50 wt %, less than or equal to 40 wt %, less than or equal to 30 wt %, or less than or equal to 20 wt % versus the total weight of the aquatic-derived biomaterial. Combinations of the above referenced ranges are also possible (for example, greater than or equal to 10 wt % and less than 100 wt %). Other ranges are also possible.

In some embodiments, as described herein the first composition comprises aquatic-derived hydroxyapatite or derivatives thereof (e.g., calcium deficient hydroxyapatite, doped hydroxyapatite, and/or substituted hydroxyapatite). For example, in some embodiments, at least a portion of the hydroxyapatite may be ionically substituted (e.g., wherein the lattice structure of the hydroxyapatite incorporates one or more of cationic (e.g., Sr²⁺, Mg²⁺, Zn²⁺, Na⁺, K⁺, Li⁺, Ag⁺, Fe²⁺, Mn²⁺, Cu²⁺) and/or anionic (e.g., CO₃²⁻, SiO₄⁴⁻, SeO₃²⁻, SeO₄²⁻, F⁻, Cl⁻, Br⁻, etc.) substitutions). While much of the specification below and herein refers to aquatic-derived hydroxyapatite generally, one of ordinary skill in the art would understand based upon the teachings of this specification that such portions of the specification generally also applies to derivatives of aquatic-derived hydroxyapatite and/or substituted aquatic-derived hydroxyapatite (e.g., such as amount present in the first composition), unless indicated to the contrary.

The aquatic-derived hydroxyapatite may be present in the first composition in any suitable amount. In some embodiments, the aquatic-derived hydroxyapatite is present in the first composition in an amount greater than or equal to 50 wt %, greater than or equal to 54 wt %, greater than or equal to 60 wt %, greater than or equal to 65 wt %, greater than or equal to 70 wt %, greater than or equal to 80 wt %, greater than or equal to 85 wt %, greater than or equal to 90 wt %, greater than or equal to 95 wt %, or greater than or equal to 98 wt % versus the total weight of the first composition. In some embodiments, the hydroxyapatite is present in the first composition in an amount less than or equal to 100 wt %, less than or equal to 98 wt %, less than or equal to 95 wt %, 90 wt %, less than or equal to 85 wt %, less than or equal to 80 wt %, less than or equal to 75 wt %, less than or equal to 70 wt %, less than or equal to 65 wt %, less than or equal to 60 wt %, or less than or equal to 55 wt % versus the total weight of the first composition. Combinations of the above-referenced ranges are also possible (e.g., greater than or equal to 50 wt % and less than or equal to 90 wt %, greater than or equal to 54 wt % and less than or equal to 65 wt %, greater than or equal to 50 wt % and less than or equal to 100 wt %). Other ranges are also possible.

The aquatic-derived biomaterial may have a desirable ratio of hydroxyapatite to collagen (or other scaffold) present in the biomaterial. For example, in some embodiments, a ratio of hydroxyapatite or derivative thereof to collagen may be greater than or equal to 70:30, greater than or equal to 80:20, or greater than or equal to 90:10 (e.g., less than or equal to 95:5, less than or equal to 90:10, less than or equal to 80:20, less than or equal to 70:30).

In some embodiments, the first composition comprises one or more additional components selected from the group consisting of amorphous biomaterial, β-tricalcium phosphate, calcium oxide, magnesium oxide, and β-rhenanite.

In some embodiments, the first composition comprises an amorphous biomaterial (e.g., amorphous calcium phosphate, non-crystallized calcium, non-crystallized phosphorous, etc.) in an amount greater than or equal to 5 wt %, greater than or equal to 10 wt %, greater than or equal to 15 wt %, greater than or equal to 19 wt %, greater than or equal to 20 wt %, greater than or equal to 25 wt %, greater than or equal to 30 wt %, greater than or equal to 32 wt %, or greater than or equal to 35 wt % versus the total weight of the first composition. In some embodiments, the first composition comprises the amorphous biomaterial in an amount less than or equal to 40 wt %, less than or equal to 35 wt %, less than or equal to 32 wt %, less than or equal to 30 wt %, less than or equal to 25 wt %, less than or equal to 20 wt %, less than or equal to 19 wt %, less than or equal to 15 wt %, or less than or equal to 10 wt % versus the total weight of the first composition. Combinations of the above-referenced ranges are also possible (e.g., greater than or equal to 5 wt % and less than or equal to 40 wt %, greater than or equal to 5 wt % and less than or equal to 25 wt %, greater than or equal to 19 wt % and less than or equal to 32 wt %). Other ranges are also possible.

In some embodiments, the first composition comprises β-tricalcium phosphate in an amount greater than or equal to 10 wt %, greater than or equal to 12 wt %, greater than or equal to 14 wt %, greater than or equal to 16 wt %, or greater than or equal to 18 wt % versus the total weight of the first composition. In some embodiments, the first composition comprises β-tricalcium phosphate in an amount less than or equal to 20 wt %, less than or equal to 18 wt %, less than or equal to 16 wt %, less than or equal to 14 wt %, or less than or equal to 12 wt % versus the total weight of the first composition. Combinations of the above-referenced ranges are also possible (e.g., greater than or equal to 10 wt % and less than or equal to 20 wt %, greater than or equal to 12 wt % and less than or equal to 16 wt). Other ranges are also possible.

In some embodiments, the first composition comprises calcium oxide, if present, in an amount greater than or equal to 0.1 wt %, greater than or equal to 0.5 wt %, greater than or equal to 1 wt %, greater than or equal to 1.5 wt %, greater than or equal to 2 wt %, greater than or equal to 2.5 wt %, greater than or equal to 3 wt %, greater than or equal to 3.5 wt %, or greater than or equal to 4 wt % versus the total weight of the first composition. In some embodiments, the first composition comprises calcium oxide in an amount less than or equal to 5 wt %, less than or equal to 4 wt %, less than or equal to 3.5 wt %, less than or equal to 3 wt %, less than or equal to 2.5 wt %, less than or equal to 2 wt %, less than or equal to 1.5 wt %, less than or equal to 1 wt %, or less than or equal to 0.5 wt % versus the total weight of the first composition. Combinations of the above-referenced ranges are also possible (e.g., greater than or equal to 0.1 wt % and less than or equal to 5 wt %, greater than or equal to 1 wt % and less than or equal to 2 wt %). Other ranges are also possible.

In some embodiments, the first composition comprises magnesium oxide, if present, in an amount greater than or equal to 0.1 wt %, greater than or equal to 0.5 wt %, greater than or equal to 1 wt %, greater than or equal to 1.5 wt %, greater than or equal to 2 wt %, greater than or equal to 2.5 wt %, greater than or equal to 3 wt %, greater than or equal to 3.5 wt %, greater than or equal to 4 wt %, greater than or equal to 5 wt %, greater than or equal to 10 wt %, or greater than or equal to 15 wt % versus the total weight of the first composition. In some embodiments, the first composition comprises magnesium oxide in an amount less than or equal to 20 wt %, less than or equal to 15 wt %, less than or equal to 10 wt %, less than or equal to 5 wt %, less than or equal to 4 wt %, less than or equal to 3.5 wt %, less than or equal to 3 wt %, less than or equal to 2.5 wt %, less than or equal to 2 wt %, less than or equal to 1.5 wt %, less than or equal to 1 wt %, or less than or equal to 0.5 wt % versus the total weight of the first composition. Combinations of the above-referenced ranges are also possible (e.g., greater than or equal to 0.1 wt % and less than or equal to 5 wt %, greater than or equal to 1 wt % and less than or equal to 2 wt %, greater than or equal to 0.1 wt % and less than or equal to 20 wt %). Other ranges are also possible.

In some embodiments, the first composition comprises β-rhenanite, if present, in an amount greater than or equal to 0.1 wt %, greater than or equal to 0.5 wt %, greater than or equal to 1 wt %, greater than or equal to 1.5 wt %, greater than or equal to 2 wt %, greater than or equal to 2.5 wt %, greater than or equal to 3 wt %, greater than or equal to 3.5 wt %, greater than or equal to 4 wt %, greater than or equal to 5 wt %, greater than or equal to 10 wt %, or greater than or equal to 15 wt % versus the total weight of the first composition. In some embodiments, the first composition comprises β-rhenanite in an amount less than or equal to 20 wt %, less than or equal to 15 wt %, less than or equal to 10 wt %, less than or equal to 5 wt %, less than or equal to 4 wt %, less than or equal to 3.5 wt %, less than or equal to 3 wt %, less than or equal to 2.5 wt %, less than or equal to 2 wt %, less than or equal to 1.5 wt %, less than or equal to 1 wt %, or less than or equal to 0.5 wt % versus the total weight of the first composition. Combinations of the above-referenced ranges are also possible (e.g., greater than or equal to 0.1 wt % and less than or equal to 5 wt %, greater than or equal to 1 wt % and less than or equal to 2 wt %, greater than or equal to 0.1 wt % and less than or equal to 20 wt %). Other ranges are also possible.

In some embodiments, the first composition has one or more desirable properties including, for example, density, thermostability, Ca/P ratio, maximum compression force, cell viability, cell proliferation, osteoinductiveness, porosity, absorbption, and/or moldability.

For example, in some embodiments, the first composition has a density similar to that of pure hydroxyapatite. In some embodiments, the density of the first composition is greater than or equal to 2 g/cm³, greater than or equal to 2.2 g/cm³, greater than or equal to 2.4 g/cm³, greater than or equal to 2.6 g/cm³, greater than or equal to 2.8 g/cm³, greater than or equal to 3 g/cm³, greater than or equal to 3.1 g/cm³, greater than or equal to 3.15 g/cm³, greater than or equal to 3.2 g/cm³, greater than or equal to 3.25 g/cm³, greater than or equal to 3.3 g/cm³, greater than or equal to 3.4 g/cm³, greater than or equal to 3.5 g/cm³, greater than or equal to 3.6 g/cm³, greater than or equal to 3.8 g/cm³, or greater than or equal to 4 g/cm³. In some embodiments, the first composition has a density of less than or equal to 4.2 g/cm³, less than or equal to 4 g/cm³, less than or equal to 3.8 g/cm³, less than or equal to 3.6 g/cm³, less than or equal to 3.5 g/cm³, less than or equal to 3.4 g/cm³, less than or equal to 3.3 g/cm³, less than or equal to 3.2 g/cm³, less than or equal to 3.25 g/cm³, less than or equal to 3.2 g/cm³, less than or equal to 3.15 g/cm³, less than or equal to 3.1 g/cm³, less than or equal to 3 g/cm³, less than or equal to 2.8 g/cm³, less than or equal to 2.6 g/cm³, less than or equal to 2.4 g/cm³, or less than or equal to 2.2 g/cm³. Combinations of the above-referenced ranges are also possible (e.g., greater than or equal to 2 g/cm³ and less than or equal to 4.2 g/cm³, greater than or equal to 3 g/cm³ and less than or equal to 3.4 g/cm³). Other ranges are also possible. Density as described herein may be measured using gas pycnometry.

In some embodiments, the first composition may be thermally stable over a desired range of temperature. For example, in some embodiments, the biomaterial is thermally stable (e.g., demonstrates no substantial mass loss) at a temperature greater than or equal to 0 °C, greater than or equal to 10 °C, greater than or equal to 25 °C, greater than or equal to 50 °C, greater than or equal to 100 °C, greater than or equal to 150 °C, greater than or equal to 200 °C, greater than or equal to 300 °C, greater than or equal to 400 °C, greater than or equal to 500 °C, greater than or equal to 600 °C, greater than or equal to 700 °C, greater than or equal to 800 °C, or greater than or equal to 900 °C. In some embodiments, the first composition is thermally stable at a temperature of less than or equal to 1000 °C, less than or equal to 900 °C, less than or equal to 800 °C, less than or equal to 700 °C, less than or equal to 600 °C, less than or equal to 500 °C, less than or equal to 400 °C, less than or equal to 300 °C, less than or equal to 200 °C, less than or equal to 150 °C, less than or equal to 100 °C, less than or equal to 50 °C, less than or equal to 25 °C, or less than or equal to 10 °C. Combinations of the above-referenced ranges are also possible (e.g., greater than or equal to 0 °C and less than or equal to 1000 °C, greater than or equal to 50 °C and less than or equal to 800 °C). That is to say, the first composition may be thermally stable over a relatively large range of temperatures. Other ranges are also possible. Thermal stability may be measured using thermogravimetric analysis.

In some embodiments, the first composition has a desirable calcium to phosphorous ratio (Ca/P) (e.g., similar to that of the stoichiometric value of hydroxyapatite). In some embodiments, the Ca/P ratio of the first composition is greater than or equal to 1.5, greater than or equal to 1.55, greater than or equal to 1.6 greater than or equal to 1.65, greater than or equal to 1.7, or greater than or equal to 1.75. In some embodiments, the Ca/P ratio of the first composition is less than or equal to 1.8, less than or equal to 1.75, less than or equal to 1.7, less than or equal to 1.65, less than or equal to 1.6, or less than or equal to 1.55. Combinations of the above-referenced ranges are also possible (e.g., greater than or equal to 1.5 and less than or equal to 1.8, greater than or equal to 1.6 and less than or equal to 1.75). Other ranges are also possible. Ca/P ratio as described herein may be measured using scanning electron microscopy coupled with energy dispersive x-ray spectroscopy (SEM-EDX).

In some embodiments, the first composition has desirable mechanical properties. For example, in some embodiments, the first composition has a maximum compression force of greater than or equal to 250 N, greater than or equal to 275 N, greater than or equal to 300 N, greater than or equal to 325 N, greater than or equal to 350 N, greater than or equal to 375 N, greater than or equal to 400 N, or greater than or equal to 500 N. In some embodiments, the first composition has a maximum compression force of less than or equal to 600 N, less than or equal to 500 N, less than or equal to 400 N, less than or equal to 375 N, less than or equal to 350 N, less than or equal to 325 N, or less than or equal to 300 N. Combinations of the above-referenced ranges are also possible (e.g., greater than or equal to 250 N and less than or equal to 600 N). Other ranges are also possible. Maximum compression force as used herein may be measured using a TA.XT plus texture analyzer to determine compression resistance.

In some embodiments, the second composition is present in the aquatic-derived biomaterial in an amount greater than or equal to 0.1 wt %, greater than or equal to 1 wt %, greater than or equal to 5 wt %, greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, or greater than or equal to 80 wt % versus the total weight of the aquatic derived biomaterial. In some embodiments, the second composition is present in the aquatic-derived biomaterial in an amount less than or equal to 90 wt %, less than or equal to 85 wt %, less than or equal to 80 wt %, less than or equal to 70 wt %, less than or equal to 60 wt %, less than or equal to 50 wt %, less than or equal to 40 wt %, less than or equal to 30 wt %, less than or equal to 20 wt %, less than or equal to 10 wt %, less than or equal to 5 wt %, less than or equal to 3 wt %, less than or equal to 2 wt %, less than or equal to 1 wt %, or less than or equal to 0.5 wt % versus the total weight of the aquatic-derived biomaterial. Combinations of the above referenced ranges are also possible (for example, greater than or equal to 0.1 wt % and less than 90 wt %). Other ranges are also possible.

In some embodiments, the second composition comprises one or more scaffold material such as a protein scaffold. Non-limiting examples of suitable scaffold materials include collagen (e.g., one or more of collagen Type I, collagen Type II, collagen Type III, collagen Type IV) and derivatives thereof, synthetic collagen and derivatives thereof, gelatin and derivatives thereof, fibrin and derivatives thereof, fibroin and derivatives thereof, fibronectin and derivatives thereof, and hydrocolloids (e.g., guar gum, arabic gum, gellan gum, xanthan gum, agar, pectin, starch, carrageenan, pectin and/or alginate) and derivatives thereof. In an exemplary set of embodiments, the second composition comprises collagen and/or derivatives thereof. In an exemplary set of embodiments, the scaffold material is aquatic-derived (e.g., fresh water derived, marine derived). In some embodiments, the scaffold material is derived from a bovine, human, equine, chicken, synthetic and/or swine source. In an exemplary set of embodiments, the scaffold material comprises salmon-derived collagen. In another exemplary set of embodiments, the scaffold material comprises bovine-derived collagen.

In some embodiments, the second composition further comprises a porogenic additive (e.g., a porogen such as an oversaturated salt solution (e.g., comprising an alkali, alkali earth material and halide, inorganic partially neutralized acid, neutralized organic acid) and/or an oil) and/or a bone morphogenetic protein.

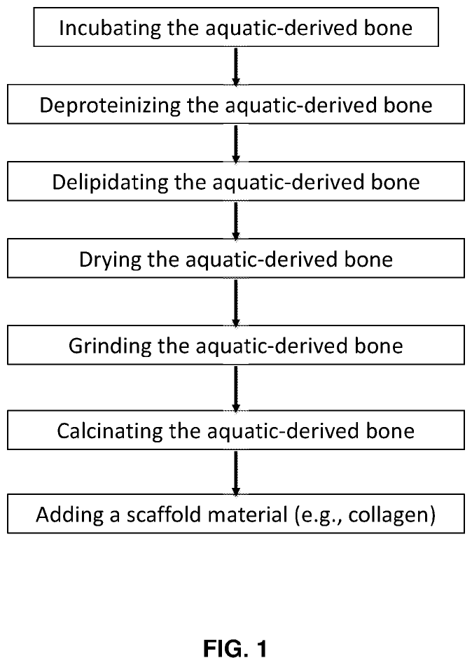

In some embodiments, the aquatic-derived biomaterial is produced using a method comprising a combination of steps including initial incubation of an aquatic-derived bone, deproteinizing the aquatic-derived bone, delipidating the aquatic-derived bone, and decalcinating the aquatic-derived bone. In some embodiments, one or more additional steps may be performed (e.g., drying and/or grinding the aquatic-derived bone). While the embodiments described herein present the steps in a particular order, other arrangements/orders of the steps may also be possible. In an exemplary method, as shown illustratively in FIG. 1, the aquatic-derived bone (e.g., salmon bone) may be first incubated, deproteinized, delipidated, dried, ground, calcinated, and a scaffold material may be added thereby forming the aquatic-derived biomaterial.

In some embodiments, non-bone material (e.g., muscle, connective tissues, amongst others) is removed from the aquatic-derived bone. For example, in some embodiments, the aquatic-derived bone is first incubated for greater than or equal to 20 minutes, greater than or equal to 30 minutes, greater than or equal to 1 hour, greater than or equal to 2 hours, greater than or equal to 3 hours, or greater than or equal to 3.5 hours thereby removing at least a portion of the non-bone material. In some embodiments, the aquatic-derived bone is first incubated for less than or equal to 4 hours, less than or equal to 3.5 hours, less than or equal to 3 hours, less than or equal to 2.5 hours, less than or equal to 2 hours, or less than or equal to 1 hour. Combinations of the above-referenced ranges are also possible (e.g., greater than or equal to 20 minutes and less than or equal to 4 hours, greater than or equal to 1 hour and less than or equal to 2 hours). Other ranges and durations are also possible.

In some embodiments, the first incubation step is performed at a temperature of greater than or equal to 70 °C, greater than or equal to 80 °C, greater than or equal to 90 °C, greater than or equal to 100 °C, greater than or equal to 110 °C, or greater than or equal to 120 °C. In some embodiments, the first incubation step is performed at a temperature of less than or equal to 120 °C, less than or equal to 110 °C, less than or equal to 100 °C, less than or equal to 90 °C, or less than or equal to 80 °C. Combinations of the above-referenced range are also possible (e.g., greater than or equal to 70 °C and less than or equal to 120 °C). Other ranges and temperatures are also possible.

In some embodiments, the first incubation step is performed in air or an aqueous solution (e.g., water, saline). In an exemplary set of embodiments, removing non-bone material comprises incubating an aquatic-derived bone for greater than or equal to 20 minutes to less than or equal to 4 hours at a temperature of greater than or equal to 70 °C and less than or equal to 120 °C thereby removing non-bone material.

In some embodiments, the aquatic-derived bone is deproteinized (e.g., after the first incubation step). In some embodiments, the deproteinizing step comprises incubating the aquatic-derived bone in an alkaline solution. Non-limiting examples of alkaline solutions include sodium hydroxide, potassium hydroxide, sodium bicarbonate, and magnesium hydroxide. Those of ordinary skill in the art would be capable of selecting suitable alkaline solutions based upon the teachings of this specification.

In some embodiments, the deproteinizing step is performed for greater than or equal to greater than or equal to 30 minutes, greater than or equal to 1 hour, greater than or equal to 2 hours, greater than or equal to 3 hours, or greater than or equal to 3.5 hours. In some embodiments, the deproteinizing step is performed for less than or equal to 4 hours, less than or equal to 3.5 hours, less than or equal to 3 hours, less than or equal to 2.5 hours, less than or equal to 2 hours, or less than or equal to 1 hour. Combinations of the above-referenced ranges are also possible (e.g., greater than or equal to 30 minutes and less than or equal to 4 hours, greater than or equal to 1 hour and less than or equal to 2 hours). Other ranges and durations are also possible.

In some embodiments, the deproteinizing step is performed at a temperature of greater than or equal to 15 °C, greater than or equal to 16 °C, greater than or equal to 18 °C, greater than or equal to 20 °C, greater than or equal to 22 °C, or greater than or equal to 24 °C. In some embodiments, the deproteinizing step is performed at a temperature of less than or equal to 25 °C, less than or equal to 24 °C, less than or equal to 22 °C, less than or equal to 20 °C, less than or equal to 18 °C, or less than or equal to 16 °C. Combinations of the above-referenced range are also possible (e.g., greater than or equal to 15 °C and less than or equal to 25 °C). Other ranges and temperatures are also possible.

In an exemplary set of embodiments, deproteinizing the aquatic-derived bone comprises incubating the aquatic-derived bone in sodium hydroxide at a temperature of greater than or equal to 15 °C and less than or equal to 25 °C for greater than or equal to 30 minutes to less than or equal to 4 hours, thereby deproteinizing the bone.

In some embodiments, the aquatic-derived bone is delipidated (e.g., after the first incubation step, after the deproteinizing step). In some embodiments, delipidating the aquatic-derived bone comprises incubating the aquatic-derived bone in a dilipidation solvent. Non-limiting examples of suitable dilipidation solvents including acetone, hexane, heptane, methanol, ethanol, isopropanol, butanol, chloroform, ether, and combinations thereof. In an exemplary set of embodiments, the dilipidation solvent is acetone, hexane, or a mixture thereof.

In some embodiments, the dilipidation step is performed for greater than or equal to greater than or equal to 4 hours, greater than or equal to 5 hour, greater than or equal to 6 hours, greater than or equal to 7 hours, greater than or equal to 8 hours, greater than or equal to 9 hours, greater than or equal to 10 hours, greater than or equal to 11 hours, greater than or equal to 12 hours, or greater than or equal to 13 hours. In some embodiments, the dilipidation step is performed for less than or equal to 14 hours, less than or equal to 13 hours, less than or equal to 12 hours, less than or equal to 11 hours, less than or equal to 10 hours, less than or equal to 9 hours, less than or equal to 8 hours, less than or equal to 7 hours, less than or equal to 6 hours, or less than or equal to 5 hours. Combinations of the above-referenced ranges are also possible (e.g., greater than or equal to 4 hours and less than or equal to 12 hours, greater than or equal to 6 hours and less than or equal to 12 hours, greater than or equal to 4 hours and less than or equal to 8 hours). Other ranges and durations are also possible.

In some embodiments, the dilipidation step is performed at a temperature of greater than or equal to 15 °C, greater than or equal to 16 °C, greater than or equal to 18 °C, greater than or equal to 20 °C, greater than or equal to 22 °C, or greater than or equal to 24 °C. In some embodiments, the dilipidation step is performed at a temperature of less than or equal to 25 °C, less than or equal to 24 °C, less than or equal to 22 °C, less than or equal to 20 °C, less than or equal to 18 °C, or less than or equal to 16 °C. Combinations of the above-referenced range are also possible (e.g., greater than or equal to 15 °C and less than or equal to 25 °C). Other ranges and temperatures are also possible.

In an exemplary set of embodiments, delipidating the aquatic-derived bone comprises incubating the aquatic-derived bone in acetone at a temperature of greater than or equal to 15 °C and less than or equal to 30 °C for greater than or equal to 4 hours to less than or equal to 8 hours, thereby delipidating the bone.

In some embodiments, the aquatic-derived bone is dried (e.g., after the deproteinizing step and/or the dilapidation step is performed). In some embodiments, the drying step is conducted to advantageously remove any remaining moisture in the aquatic-derived bone. In some embodiments, the drying step is performed for greater than or equal to 4 hours, greater than or equal to 6 hours, greater than or equal to 8 hours, greater than or equal to 10 hours, greater than or equal to 12 hours, greater than or equal to 16 hours, greater than or equal to 20 hours, or greater than or equal to 24 hours. In some embodiments, the drying step is performed for less than or equal to 48 hours, less than or equal to 24 hours, less than or equal to 20 hours, less than or equal to 16 hours, less than or equal to 12 hours, less than or equal to 10 hours, less than or equal to 8 hours, or less than or equal to 6 hours. Combinations of the above-referenced ranges are also possible (e.g., greater than or equal to 4 hours and less than or equal to 24 hours, greater than or equal to 6 hours and less than or equal to 48 hours). Other ranges and durations are also possible.

In some embodiments, the drying step is performed at a temperature of greater than or equal to 40 °C, greater than or equal to 45 °C, greater than or equal to 50 °C, greater than or equal to 55 °C, greater than or equal to 60 °C, or greater than or equal to 65 °C. In some embodiments, the dilipidation step is performed at a temperature of less than or equal to 70 °C, less than or equal to 65 °C, less than or equal to 60 °C, less than or equal to 55 °C, less than or equal to 50 °C, or less than or equal to 45 °C. Combinations of the above-referenced range are also possible (e.g., greater than or equal to 40 °C and less than or equal to 70 °C). Other ranges and temperatures are also possible.

In an exemplary set of embodiments, the drying step comprises drying the aquatic-derived bone at a temperature of greater than or equal to 50 °C and less than or equal to 70 °C for greater than or equal to 6 hours and less than or equal to 48 hours.

In some embodiments, the aquatic-derived bone is ground (e.g., milling such as by ball milling, manual or electric grinder, mortar and pestle, or the like) to form particles of the aquatic-derived bone. In some embodiments, the grinding may be performed at any suitable temperature (e.g., room temperature, a drying temperature) for greater than or equal to 1 second, greater than or equal to 2 seconds, greater than or equal to 4 seconds, greater than or equal to 6 seconds, greater than or equal to 8 seconds, or greater than or equal to 10 seconds. In some embodiments, the aquatic-derived bone is ground for less than or equal to 12 seconds, less than or equal to 10 seconds, less than or equal to 8 seconds, less than or equal to 6 seconds, less than or equal to 4 seconds, or less than or equal to 2 seconds. Combinations of the above-referenced ranges are also possible (e.g., greater than or equal to 1 second and less than or equal to 12 seconds). Other ranges and durations are also possible.

In some embodiments, grinding the aquatic-derived bone (e.g., after the drying step) results in a plurality of particles. Advantageously, the duration of performing the grinding step may produce a plurality of particles having a particle size similar to commercially available allografts and/or xenografts. In some embodiments, the plurality of particles advantageously have a particle size sufficient to maintain a desirable porosity of the particle material.

In some embodiments, at least a portion of the plurality of particles have a particle size of greater than or equal to 250 microns and less than or equal to 1000 microns. For example, in some embodiments, an average particle size of the aquatic-derived bone after griding is greater than or equal to 100 microns, greater than or equal to 250 microns, greater than or equal to 500 microns, greater than or equal to 750 microns, or greater than or equal to 1000 microns. In some embodiments, the average particle size is less than or equal to 2000 microns, less than or equal to 1000 microns, less than or equal to 750 microns, less than or equal to 500 microns, or less than or equal to 250 microns. Combinations of the above-referenced ranges are also possible (e.g., greater than or equal to 100 microns and less than or equal to 2000 microns, greater than or equal to 250 microns and less than or equal to 1000 microns). Other ranges and sizes are also possible. Those of ordinary skill in the art would understand that while the average particle size may have a range as noted above (e.g., greater than or equal to 100 microns and less than or equal to 2000 microns), the plurality of particles may include individual particles having particle sizes less than 100 microns and/or greater than 2000 microns.

In some embodiments, the aquatic-derived bone (e.g., after the deproteinizing step, after the dilapidation step, after the drying step, and/or after the grinding step is performed) is calcinated. For example, in some embodiments, the aquatic-derived bone (e.g., aquatic-derived bone particles) is calcinated thereby producing the first composition (e.g., a first composition comprising hydroxyapatite as described herein). In some embodiments, the step of calcination comprises heating the (ground) aquatic-derived bone to a temperature of greater than or equal to 600 °C, greater than or equal to 650 °C, greater than or equal to 700 °C, greater than or equal to 800 °C, greater than or equal to 900 °C, greater than or equal to 1000 °C, or greater than or equal to 1100 °C. In some embodiments, the step of calcination comprises heating to a temperature of less than or equal to 1200 °C, less than or equal to 1100 °C, less than or equal to 1000 °C, less than or equal to 900 °C, less than or equal to 800 °C, less than or equal to 700 °C, or less than or equal to 650 °C. Combinations of the above-referenced ranges are also possible (e.g., greater than or equal to 600 °C and less than or equal to 1200 °C, greater than or equal to 800 °C and less than or equal to 1200 °C). Other ranges and temperatures are also possible.

In some embodiments, the calcination step comprises heating the (ground) aquatic-derived bone (e.g., aquatic-derived bone particles) for greater than or equal to 1 hour, greater than or equal to 1.5 hours, greater than or equal to 2 hours, greater than or equal to 2.5 hours, greater than or equal to 3 hours, greater than or equal to 4 hours, greater than or equal to 5 hours, greater than or equal to 6 hours, or greater than or equal to 8 hours. In some embodiments, the calcination step comprises heating the (ground) aquatic-derived bone (e.g., aquatic-derived bone particles) for less than or equal to 10 hours, less than or equal to 8 hours, less than or equal to 6 hours, less than or equal to 5 hours, less than or equal to 4 hours, less than or equal to 3 hours, less than or equal to 2.5 hours, less than or equal to 2 hours, or less than or equal to 1.5 hours. Combinations of the above-referenced ranges are also possible (e.g., greater than or equal to 1 hour and less than or equal to 10 hours). Other ranges and durations are also possible.

In some embodiments, after formation of the first composition, the first composition and second composition are mixed together (e.g., in a weight ratio as described above) thereby forming the aquatic-derived biomaterial. In some embodiments, the first composition and second composition are combined such that a ratio of hydroxyapatite to the second composition (e.g., collagen or a derivative thereof) present in the aquatic-derived biomaterial is greater than or equal to 70:30, greater than or equal to 75:25, greater than or equal to 80:20, greater than or equal to 85:15, or greater than or equal to 90:10. In some embodiments, the ratio of hydroxyapatite to the second composition (e.g., collagen or a derivative thereof) is less than or equal to 95:5, less than or equal to 90:10, less than or equal to 85:15, less than or equal to 80:20, or less than or equal 75:25. Combinations of the above referenced ranges are also possible (e.g., greater than or equal to 70:30 and less than or equal to 90:10). Other ranges are also possible. In an exemplary set of embodiments, the ratio of hydroxyapatite to collagen (or a derivative thereof) in the biomaterial is greater than or equal to 70:30 and less than or equal to 90:10).

In an exemplary set of embodiments, the method for forming the first composition comprises one or more of the following steps:
1. Incubating salmon bones in water at 110 °C for 1 to 2 hours. This step corresponds to the removal of substances other than bones, such as muscle, connective tissues, or other elements.
2. Incubating the salmon bones in a NaOH solution at a concentration between 0.1 M and 0.5 M, preferably between 0.2 M and 0.3 M, and more preferably at 0.25 M, at a temperature between 15 °C and 30 °C, preferably between 18 °C and 23 °C, and more preferably at 20 °C, for a time between 1 and 3 hours, preferably 2 hours. This step corresponds to the deproteinization of the bones.
3. Incubating the salmon bones in acetone between 20 °C and 30 °C, preferably between 22 °C and 28 °C, and more preferably at 25 °C, for a time between 5 and 7 hours, preferably between 5.5 and 6.5 hours, and more preferably for 6 hours. This step corresponds to the delipidation of the bones.
4. Drying the salmon bones between 50 °C and 70 °C, preferably between 55 °C and 65 °C, and more preferably at 60 °C, for more than 6 hours.
5. Grinding the bones and calcining them at a temperature between 900 °C and 1100 °C, preferably between 950 °C and 1050 °C, and more preferably at 1000 °C, for a time between 1 and 3 hours, preferably between 1.5 and 2.5 hours, and more preferably for 2 hours.
6. Obtaining the biomaterial comprising hydroxyapatite.
7. In a exemplary set of embodiments embodiment, the method additionally includes a final step in which collagen is added between 10 and 90% (w/w) of the final composition, thus obtaining the composition of the present invention.

In some embodiments, the method includes an additional step of adding platelet-rich fibrin to the aquatic-derived biomaterial comprising hydroxyapatite.

In an exemplary set of embodiments, the biomaterial comprising hydroxyapatite, low-cost, non-cytotoxic, and promoting osteogenesis, obtained using the method described above, comprises between 54 and 65% of hydroxyapatite, 12 and 16% of β-tricalcium phosphate, between 1 and 2% of calcium oxide, between 0 and 2% of magnesium oxide, and between 19 and 32% of amorphous biomaterial. The exemplary biomaterial has a density between 3.2 g/cm³ and 3.3 g/cm³ and is thermostable between 50 °C and 800 °C. Other components and ranges are also possible as described above and herein.

In an exemplary set of embodiments of the invention, the aquatic-derived biomaterial is in solid, semi-solid, or liquid form, such as a solid block, a gel, a paste, etc.

An exemplary set of embodiments is directed the use of the aquatic-derived biomaterial comprising a first composition comprising hydroxyapatite derived from salmon bone and a second composition comprising collagen in the manufacture of a product useful for promoting osteogenesis.

In an exemplary set of embodiments, the composition including the biomaterial obtained from aquatic-derived bone additionally comprises collagen, which can be type 1 collagen, type 2 collagen, type 3 collagen, and type 4 collagen. Without wishing to be bound by theory, in some embodiments, the addition of collagen (or other scaffold materials as described herein) advantageously improves the ability of the biomaterial to promote osteogenesis. The collagen may be present in the biomaterial in any suitable amount such as those described above in the context of the second composition.

Collagen is an essential protein found in the extracellular matrix of bone tissues and is crucial for bone structure and strength. Without wishing to be bound by theory, by incorporating collagen into the biomaterial, the integration of the biomaterial with the surrounding bone tissue can be improved, promoting the formation of new bone.

Additionally, collagen can provide a favorable environment for the adhesion and proliferation of bone cells, which can accelerate the healing process of bone injuries.

In another exemplary embodiment, the composition comprising the hydroxyapatite biomaterial obtained from aquatic-derived bone additionally comprises platelet-rich fibrin. Platelet-rich fibrin (PRF) is an autologous platelet concentrate obtained from the patient's own blood. PRF is rich in growth factors and other bioactive proteins that can promote tissue regeneration. By incorporating PRF into the biomaterial, the ability of the biomaterial to promote osteogenesis, its biocompatibility, and the healing of bone injuries may be improved, in some cases.

In some embodiments, the aquatic-derived biomaterial further comprises platelet rich-fibrin, if present, in an amount greater than or equal to 0.1 wt %, greater than or equal to 0.5 wt %, greater than or equal to 1 wt %, greater than or equal to 1.5 wt %, greater than or equal to 2 wt %, greater than or equal to 2.5 wt %, greater than or equal to 3 wt %, greater than or equal to 3.5 wt %, or greater than or equal to 4 wt % versus the total weight of the biomaterial. In some embodiments, the aquatic-derived biomaterial comprises platelet rich-fibrin in an amount less than or equal to 5 wt %, less than or equal to 4 wt %, less than or equal to 3.5 wt %, less than or equal to 3 wt %, less than or equal to 2.5 wt %, less than or equal to 2 wt %, less than or equal to 1.5 wt %, less than or equal to 1 wt %, or less than or equal to 0.5 wt % versus the total weight of the biomaterial. Combinations of the above-referenced ranges are also possible (e.g., greater than or equal to 0.1 wt % and less than or equal to 5 wt %, greater than or equal to 1 wt % and less than or equal to 2 wt %). Other ranges are also possible.

In yet another embodiment of the invention, the composition including the hydroxyapatite biomaterial can be in solid, semi-solid, or liquid form.

Another object of the present invention is the use of the composition including the biomaterial comprising hydroxyapatite derived from salmon bones in a product useful for promoting osteogenesis and for the treatment of bone injuries.

All technical and scientific terms used to describe the present invention have the same meaning understood by a person with basic knowledge in the relevant technical field. However, to define the scope of the invention more clearly, the following is a list of the terminology used in this description. The term “bone injuries” generally refers to a variety of conditions and disorders affecting the structural and functional integrity of bones and bone tissues, including dental injuries. These may include, but are not limited to, bone fractures, osteoporosis, stress injuries, periodontal disease, dental abscesses, peri-implantitis, osteomyelitis, bone tumors, bone metastases, among others. The term “osteogenesis” generally refers to the biological process that encompasses the formation and development of new bone in the body. It is essential in the growth stage of an individual, during the healing of fractures, and in constant bone remodeling, which allows for the maintenance and renewal of the skeleton throughout a person's life. Osteogenesis can be of two types:
intramembranous and endochondral. In intramembranous osteogenesis, bone is formed directly from condensed mesenchymal cells that differentiate into osteoblasts, which in turn generate bone. This type of osteogenesis is the main bone-forming process in the face and skull. On the other hand, endochondral osteogenesis involves the formation of bone from a cartilage template that is gradually replaced by bone tissue. This is the predominant process in the formation of long bones in the body, such as those in the arm or leg. In the dental context, osteogenesis is a key process in various dental and maxillofacial interventions. For example, in dental implant procedures, where a titanium implant is inserted into the mandibular or maxillary bone, osteogenesis is sought to be promoted around the implant to achieve solid and durable integration, a phenomenon known as osseointegration.

Additionally, in orthognathic surgeries aimed at correcting jaw deformities, cuts are made in the bone which are then fixed in a new position. During recovery, osteogenesis is the process that allows for the healing and fusion of repositioned bone segments. Finally, in guided bone regeneration procedures, which are used to treat bone loss due to periodontal disease or to prepare the ground for a future dental implant, biomaterials and techniques that stimulate osteogenesis are used to replace lost bone tissue. The term “hydroxyapatite” or “HAPS” refers to a calcium biomineral, specifically a form of calcium phosphate, with the chemical formula Ca₅ (PO₄)₃(OH). Hydroxyapatite is the main mineral component of human bones and teeth, forming approximately 70% of bone mass and up to 90% of dental enamel. It is recognized for its excellent biocompatibility and bioactivity, making it an ideal biomaterial for applications in orthopedics, dentistry, and regenerative medicine. In the form of microscopic crystals, hydroxyapatite provides hardness and structural rigidity in bones and teeth. Additionally, due to its chemical composition like that of bone, it is frequently used in implants and bone filler biomaterials, as it can promote the formation of new bone and integration with surrounding tissue. The term “osteoinduction” refers to a biological process that involves the stimulation of undifferentiated progenitor cells to differentiate into bone cells or osteoblasts. This process mainly occurs through biochemical signals emitted by bone growth factors and is crucial in the regeneration and remodeling of bone tissue. For example, osteoinduction plays a crucial role in the success of bone grafts, where the grafted biomaterial induces the formation of new bone at the graft site. The potential for differentiation into bone tissue is of great relevance and accounts for the phenomena of osteogenesis and osteoinduction, since if mesenchymal cells have the ability to differentiate into osteoblasts, which are bone tissue cells specialized in producing bone matrix, formed of hydroxyapatite crystals, and mainly composed of Ca and P, it would also be demonstrating the ability to generate these structures.

Osteoprogenitor cells present in the patient potentially can proliferate and differentiate into osteoblasts in the presence of a graft; therefore, based on the above, a biomaterial in this context may, in some embodiments, have osteogenic and osteoinductive activity, as it may be related to bone formation, in addition to stimulating and activating the mesenchymal stem cells of the host tissue, respectively.

The term “allograft” refers to a specific type of transplant in which tissue is transferred from one individual to another of the same species but is not a genetically identical twin. Allografts are common in medical practice and are used in a variety of procedures, such as organ transplants, skin tissue for treating severe burns, or in orthopedic surgeries where allogeneic bone grafts are used. Since donors and recipients are not genetically identical, it is essential to consider the risk of immune rejection, and therefore, the use of immunosuppressive drugs is often necessary to prevent this problem.

### EXAMPLES

The following examples are intended to illustrate certain embodiments described herein, including certain aspects of the present invention, but do not exemplify the full scope of the invention.

### Example 1—Optimization of Desproteinization Variables

In this example, the optimization of the variables of the production method of the present invention, focusing on the desproteinization stage, was carried out. A series of experiments were conducted to test different values of alkaline solution concentration and temperature during this stage.

### Procedure:

1. The salmon bones were initially subjected to a suspension in water at 110 °C for 2 hours to remove most of the substances other than the bones, such as muscle and connective tissues.
2. Then, the desproteinization stage was implemented, where the bones were treated with a NaOH solution at different concentrations (2 M or 0.25 M) and temperatures (20 °C or 50 °C) for a period of 2 hours.
3. After desproteinization, the bones were subjected to a delipidation stage with acetone at room temperature for 24 hours.
4. Subsequently, the bones were dried at 60 °C overnight to remove any remaining moisture.
5. Finally, the dried bones were ground in preparation for the final heating (i.e. calcination) stage, where they were transformed into a hydroxyapatite biomaterial through a process at 650 °C for 5 hours.

Results: The results of the yield based on the variables of the desproteinization stage are shown in Table 1. The combination of NaOH 2 M and 20 °C showed the most effective yield, with a yield of 12%.

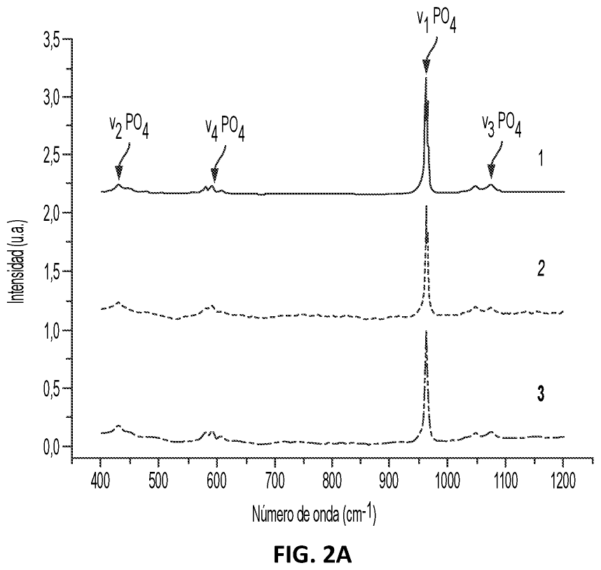

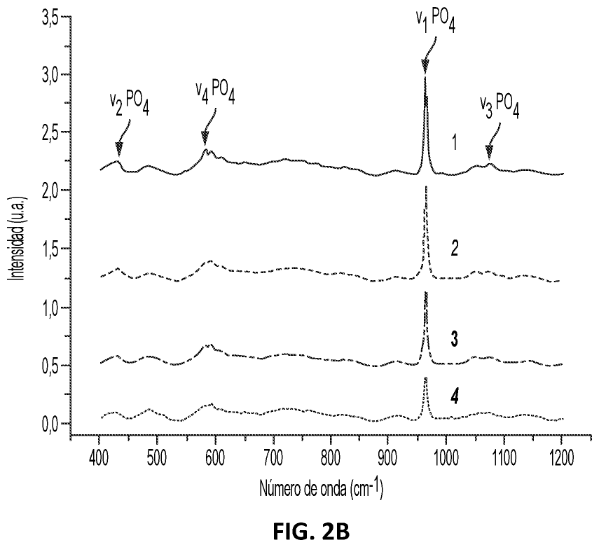

In addition, Raman spectra measurements (FIG. 2A) were made for the resulting biomaterials from each combination of conditions. The 4 vibrational modes assigned to the PO4 group of apatite were observed, with the characteristic peak of hydroxyapatite being the most relevant.

TABLE 1
Results of yield based on the variables of the deproteinization stage

| Variables | Yield (%) |
|---|---|
| NaOH 2M and 20 °C | 12.0 |
| NaOH 2M and 50 °C | 6.0 |
| NaOH 0.25M and 20 °C | 7.8 |
| NaOH 0.25M and 50 °C | — |

Conclusion: Based on the results, the treatment conditions with NaOH 2.5 M and 50 °C were discarded, as they did not show adequate yield. The combination of NaOH 2 M and 20 °C was identified as the most effective for the desproteinization of salmon bones. This example illustrates the process of optimizing variables in the desproteinization stage, crucial for obtaining high-quality and high-yield hydroxyapatite biomaterial.

### Example 2—Optimization of Delipidation Variables

The variables of the production method of the present invention, particularly the solvent and time in the delipidation stage, were determined through a series of experiments testing different values for each variable.

### Procedure:

1. Salmon bones were initially subjected to a suspension in water at 110 °C for 2 hours.
2. Subsequently, the desproteinization stage was implemented, where the bones were treated with a NaOH solution at a concentration of 2 M. This process was carried out at a constant temperature of 20 °C for a period of 2 hours.
3. After desproteinization, the bones underwent a delipidation stage with acetone or hexane at 25 °C for 6 or 12 hours.
4. The bones were then dried at 60 °C overnight to remove any remaining moisture.
5. Finally, the dried bones were ground in preparation for the final calcination stage, where they were transformed into a hydroxyapatite biomaterial through a process at 650 °C for 5 hours.

Results: The results of the yield based on the variables of the delipidation stage are shown in Table 2. The combination of acetone and 6 hours showed the most effective yield at 17.8%.

TABLE 2
Results of yield based on the variables of the delipidation stage

| Variables | Yield (%) |
|---|---|
| Acetone and 6 hours | 17.8 |
| Acetone and 12 hours | 17.7 |
| Hexane and 6 hours | 16.0 |
| Hexane and 12 hours | 17.0 |

Additionally, Raman spectra measurements were made for the resulting biomaterials from each combination of conditions using an XploRA™ PLUS equipment (Horiba Scientific, France), as shown in FIG. 2B. While no marked effects related to time were observed, a peak of intensity at 910 cm-1 was more pronounced for experiments with hexane, and a less intense hydroxyapatite peak was observed compared to samples with acetone.

### Example 3—Optimization of Calcination Variables

The variables of the production method of the present invention, particularly the temperature and time in the calcination stage, were determined through a series of experiments testing different values for each variable.

### Procedure:

1. Salmon bones were initially subjected to a suspension in water at 110 °C for 2 hours.
2. Subsequently, the desproteinization stage was implemented, where the bones were treated with a NaOH solution at a concentration of 2 M. This process was carried out at a constant temperature of 20 °C for a period of 2 hours.
3. After desproteinization, the bones underwent a delipidation stage with acetone at 25 °C for 6 hours.
4. The bones were then dried at 60 °C overnight to remove any remaining moisture.
5. Finally, the dried bones were ground in preparation for the final calcination stage, where they were transformed into a hydroxyapatite biomaterial through a process at 800 °C, 900 °C, or 1000 °C for 2, 4, or 6 hours.

The results of the yield based on the variables of the calcination stage are shown in Table 3. The combination of 800 °C and 2 hours showed the most effective yield at 19.0%.

TABLE 3
Results of yield based on the variables of the calcination stage

| Variables | Yield (%) |
|---|---|
| 800 °C and 6 hours | 18.9 |
| 800 °C and 4 hours | 18.9 |
| 800 °C and 2 hours | 19.0 |
| 900 °C and 6 hours | 18.0 |
| 900 °C and 4 hours | 18.0 |
| 900 °C and 2 hours | 18.8 |
| 1000 °C and 6 hours | 18.0 |
| 1000 °C and 4 hours | 18.0 |
| 1000 °C and 2 hours | 18.0 |

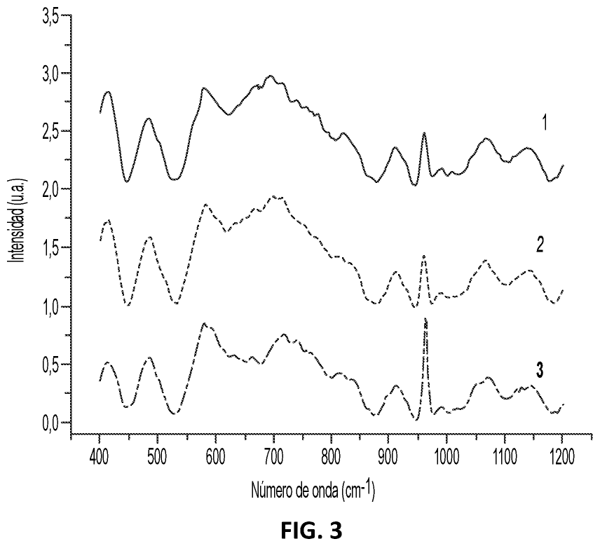

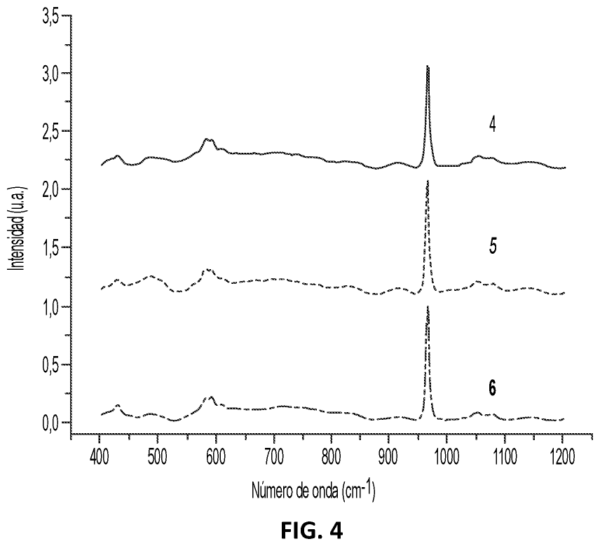

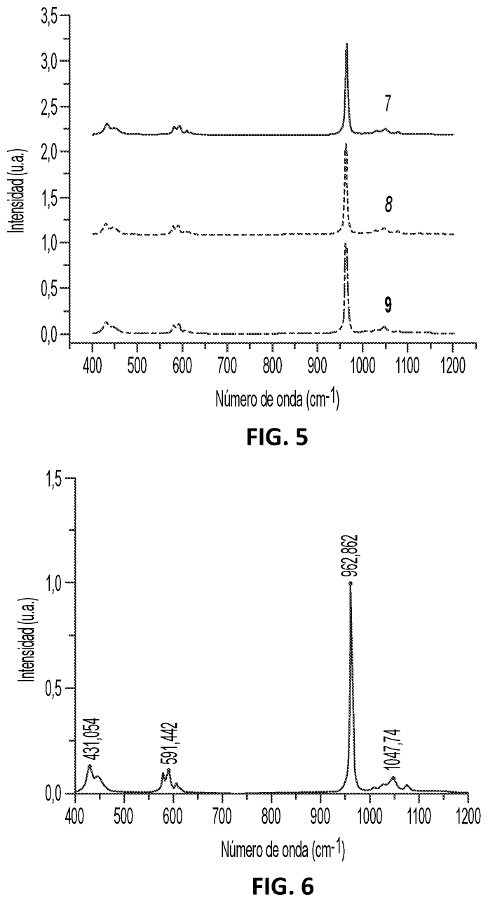

Additionally, Raman spectra measurements were made for the resulting biomaterials from each combination of conditions using an XploRA™ PLUS equipment (Horiba Scientific, France), as shown in FIGS. 3, 4, and 5.

The characteristic maximum intensity of hydroxyapatite was identified in all samples at the previously reported position. The time variable did not seem to influence the formation of hydroxyapatite, as the spectra showed no notable differences at a given temperature, and all of them exhibited the characteristic peaks of the hydroxyapatite structure. However, temperature did influence the purity of hydroxyapatite, with a more defined spectrum observed at higher temperatures.

### Example 4—Selected Protocols

Three complete protocols were evaluated as follows:
Protocol 1: Desproteinization 0.25 M at 20 °C for 2 hours/Delipidation with acetone for 6 hours/Calcination at 1000 °C for 2 hours.
Protocol 2: Desproteinization 0.25 M at 20 °C for 2 hours/Delipidation with acetone for 6 hours/Calcination at 900 °C for 2 hours.
Protocol 3: Desproteinization 0.25 M at 20 °C for 2 hours/Delipidation with acetone for 6 hours/Calcination at 800 °C for 2 hours.

From the above protocols, the results shown in Table 4 were obtained, where the biomaterials obtained from the 3 tested protocols were selected as equivalent in quality.

TABLE 4
Results of the 3 complete protocols
to obtain hydroxyapatite biomaterial.

| | | Hydroxyapatite | |
|---|---|---|---|
| Protocol | Yield (%) | Amount (% w/w) | Crystallinity (%) |
| 1 | 19.0 | 64.1 | 80.49 |
| 2 | 21.0 | 60.21 | 78.45 |
| 3 | 18.9 | 54.33 | 68.42 |

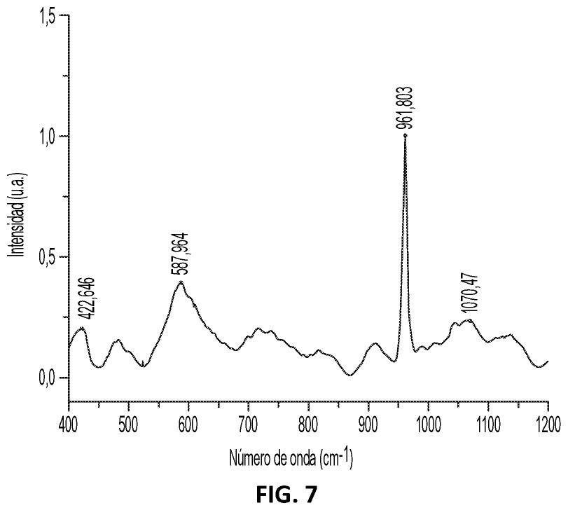

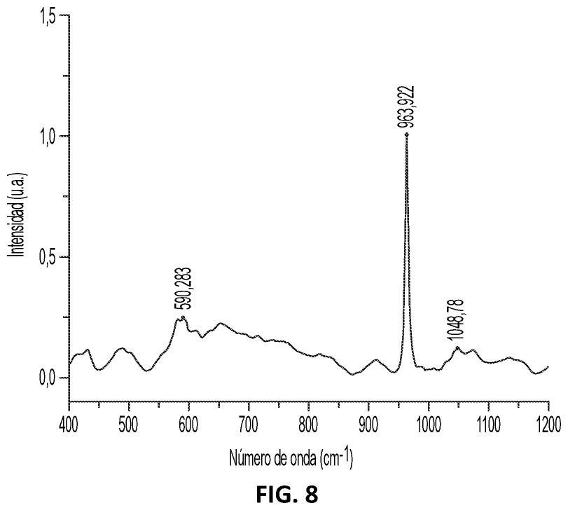

Raman spectra were then measured for the resulting biomaterials from each protocol using an XploRA™ PLUS equipment (Horiba Scientific, France), as shown in FIGS. 6, 7, and 8.

The 4 vibrational modes assigned to the phosphate group (PO4) of apatite were presented in each of the spectra, with sharper peaks corresponding to hydroxyapatite produced by protocol 1, while for the biomaterial from protocols 2 and 3, noisy forms were exhibited, along with the peaks of the apatite structure. The peak appearing in the 1070 cm-1 region corresponds to the carbonate group peak that overlaps with the phosphate group v3. The presence of the carbonate group in bone graft tissues is important, as it is described as the factor that simulates the extracellular matrix, thus improving osteointegration, biocompatibility, and resorption characteristics.

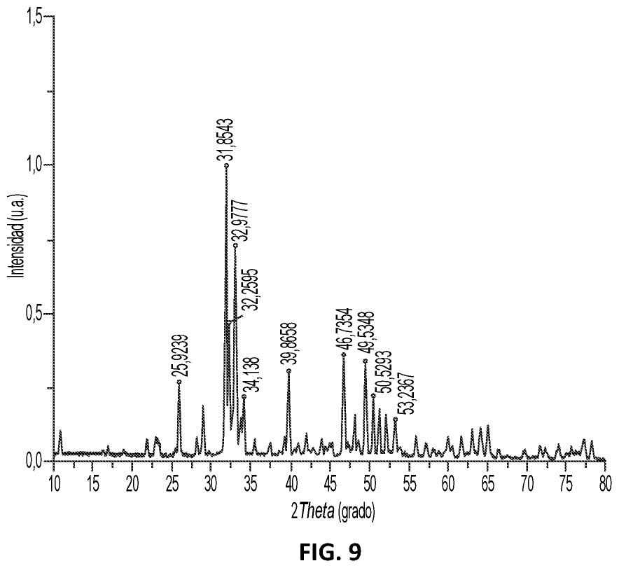

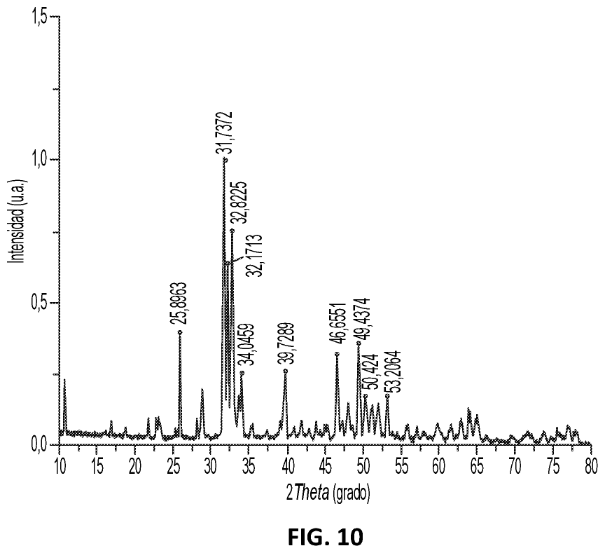

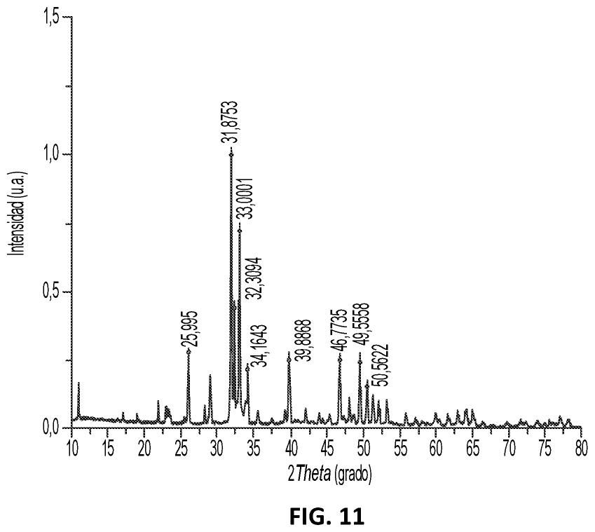

X-ray diffraction (XRD) was then measured for the resulting biomaterials from each of the 3 protocols using a powder X-ray diffractometer (Bruker, Germany). The obtained diffractograms can be seen in FIGS. 9, 10, and 11, translating into the results shown in Table 5.

TABLE 5
Results of the diffractograms of the biomaterials obtained from the 3 protocols.

| | | β- | | | |
|---|---|---|---|---|---|
| | | Tricalcium | Calcium | Magnesium | Amorphous |
| | Hydroxyapatite | phosphate | oxide (% | oxide (% | material (% |
| Protocol | (% w/w) | (% w/w) | w/w) | w/w) | w/w) |
| 1 | 64.10 | 15.40 | 0.99 | — | 19.51 |
| 2 | 60.21 | 14.33 | 1.91 | 2.00 | 21.55 |
| 3 | 54.33 | 12.62 | 1.33 | 0.14 | 31.58 |

### Example 5—Thermogravimetric Analysis (TGA)

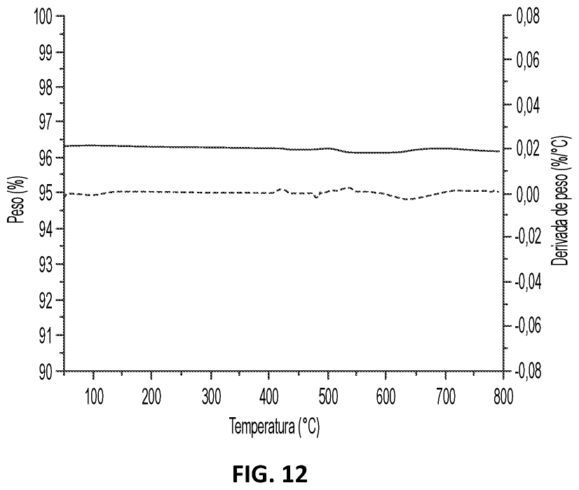

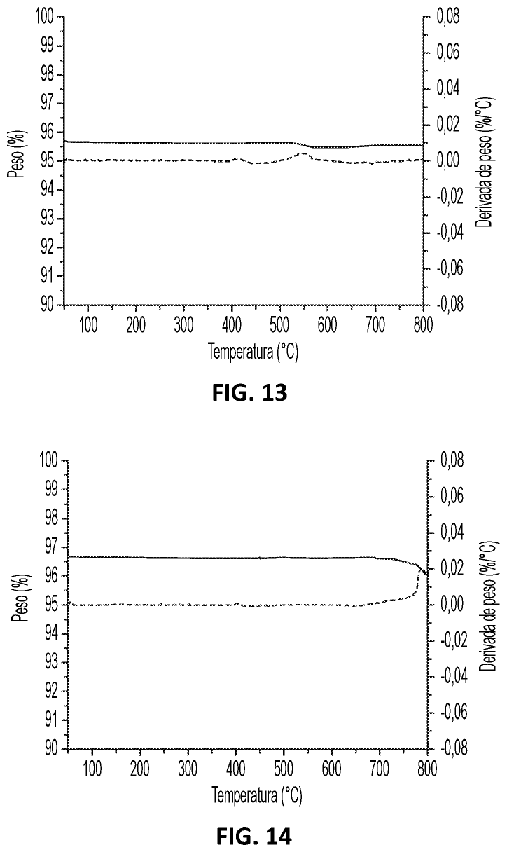

Thermogravimetric analysis (TGA) was performed on the biomaterials resulting from protocols 1, 2, and 3 using a TGA Q50 V20.10 Build 36 equipment (Thermal Analysis, United States), varying the temperature between 50 °C and 800 °C. The results for each protocol can be seen in FIGS. 12, 13, and 14. For the hydroxyapatite of the selected protocols, no significant mass loss is evidenced. However, a small variation can be noted from the temperature of 500 °C to 600 °C, both for protocol 1 and protocol 2, which could indicate the combustion of traces of remaining organic matter in the biomaterial. This confirms that the produced hydroxyapatite is thermally stable.

### Example 6—Scanning Electron Microscopy, Coupled with Energy Dispersive X-Ray Spectroscopy (SEM-EDX)

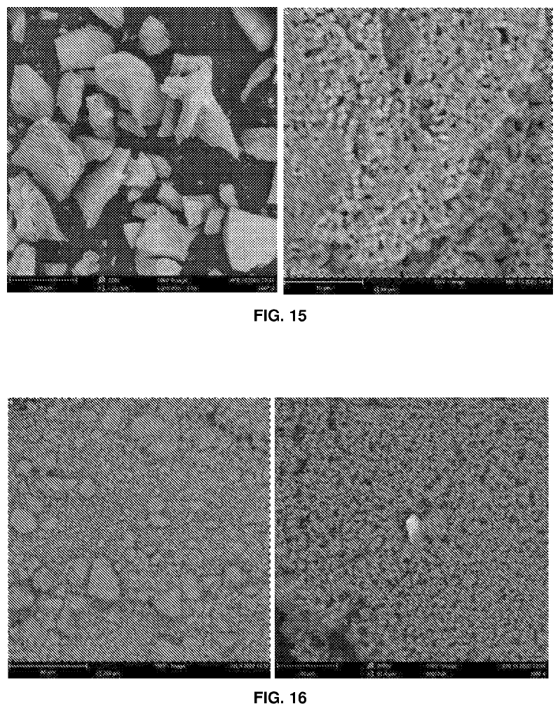

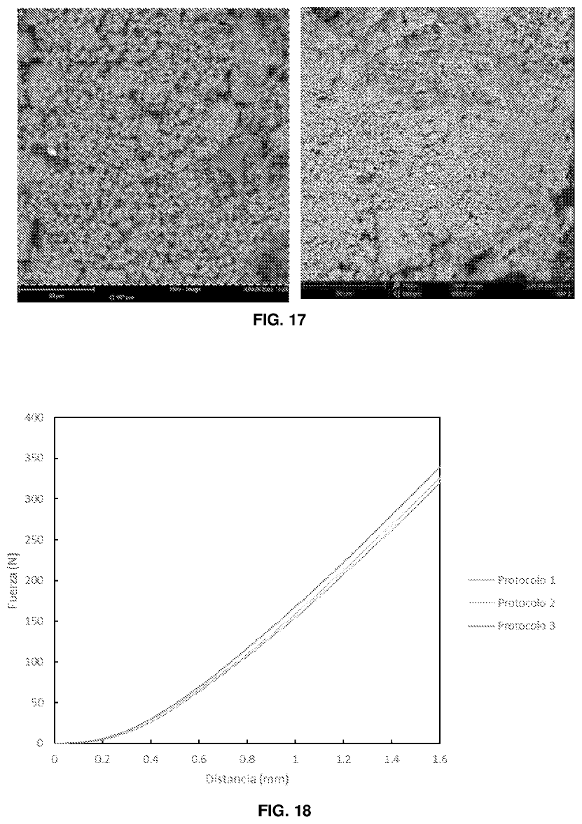

The resulting biomaterials from protocols 1, 2, and 3 were sent for SEM-EDX microscopy using a Phenom Pro X equipment (Thermo Fisher Scientific, United States). The obtained images can be observed in FIGS. 15, 16, and 17. Additionally, the local elemental composition, extracted from the EDX analysis, is presented in Table 6.

TABLE 6
Local elemental composition of the biomaterials
obtained from each protocol.

| Chemical | | | |
|---|---|---|---|
| Symbol/Atomic | | | |
| Concentration | | | |
| (% w/w) | Protocol 1 | Protocol 2 | Protocol 3 |
| Ca | 7.64 | 18.20 | 18.87 |
| P | 4.68 | 10.46 | 10.84 |
| Ca/P | 1.63 | 1.74 | 1.74 |

The Ca/P ratio of the obtained hydroxyapatites is close to the stoichiometric value of hydroxyapatite, which is 1.67. In addition to the elements in Table 5, quantities of C, O, Mg, Na, N, Cu, Al, and Si were presented.

### Example 7—Gas Pycnometry

Gas pycnometry analysis was performed on the biomaterials obtained by each of the 3 selected protocols using an AccuPyc II 1340 Series equipment (Micromeritics, United States). The measured densities can be observed in Table 7.

TABLE 7
Density of the biomaterials obtained from each extraction protocol.

| Protocol | Density (g/cm3) |
|---|---|
| 1 | 3.24 ± 1.10 |
| 2 | 3.29 ± 0.01 |
| 3 | 3.37 ± 0.01 |

The obtained values are close to the theoretical density for pure hydroxyapatite of 3.156 g/cm³.

### Example 8—Texture Analyzer

The compression resistance of the samples from each protocol was analyzed using a TA.XT Plus Texture Analyzer (Stable Micro Systems, United Kingdom), obtaining the slope as shown in FIG. 18. Based on the data obtained, 6 replicates were considered for each protocol. The measurements were performed with compression force up to 2 mm. The calculations of the slope and determination of maximum force were made considering the data from 0.5 to 1.6 mm of compression, and they can be seen in Table 8.

TABLE 8
Maximum compression force of the biomaterials
obtained from each extraction protocol.

| Protocol | Maximum Force (N) |
|---|---|
| 1 | 320.60 |
| 2 | 326.26 |
| 3 | 339.55 |

These results indicate that at lower density (material from protocol 1), and therefore higher porosity, there was less mechanical resistance than the others. Therefore, without wishing to be bound by theory, higher temperatures in the calcination process of hydroxyapatite production may, in some cases, interfere with the resulting structure of the final biomaterial, affecting its compression resistance by presenting higher porosity.

### Example 9—Cell Viability and Proliferation

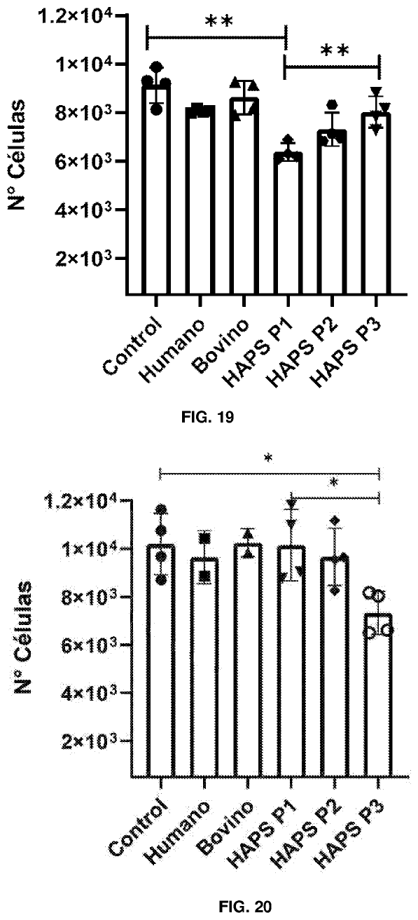

To assess the viability and proliferation of different cell types in the presence of hydroxyapatite biomaterials obtained from the 3 selected protocols, gingival mesenchymal cells were cultured in Dulbecco's Modified Eagle Medium (DMEM) for 48 hours in the presence of commercial hydroxyapatite biomaterials of human origin, bovine origin, originating from each of the 3 selected protocols, and a negative control. Viability and proliferation were then measured using 3-(4,5-dimethylthiazol-2-yl)-2,5-diphenyltetrazolium bromide (MTT) assay to measure metabolically active cells. The results at 24 and 48 hours of culture can be seen in FIGS. 19 and 20.

The number of viable cells was similar in all tested conditions, except for the hydroxyapatite biomaterial from protocol 1 (HAPS P1), which showed significant differences compared to the control and hydroxyapatite biomaterial from protocol 3 (HAPS P3) (p<0.01) at 24 h. Then, in FIG. 20, at 48 hours, the cell viability of HAPS changes, observing that the cells treated with HAPS P3 do not increase in number compared to the 24 h experiment, while the other conditions do increase their viability, reaching a similar number of cells among them.

Although HAPS P3 shows significant differences compared to the controls and other hydroxyapatites at 48 h, the number of viable cells remains acceptable, suggesting that hydroxyapatite produced from salmon bone, considered a waste of the salmon industry, has value for the field of bone repair.

### Example 10—Cellular Differentiation of Mesenchymal Cells into Osteoblasts

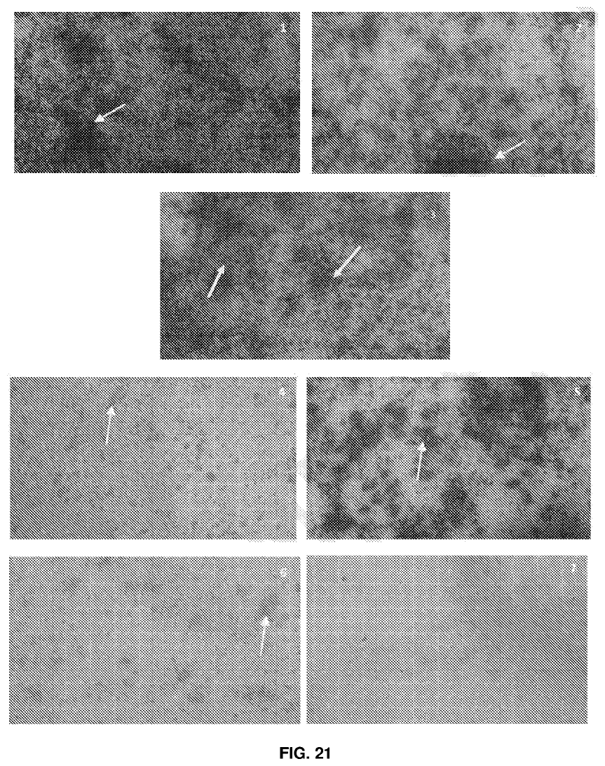

Mesenchymal cells were cultured for a 21-day differentiation period, which were stained with alizarin red in differentiation medium (DMEM supplemented with ascorbic acid, sodium phosphate, and dexamethasone) at 37 °C. Differentiations were performed under different conditions, particularly in the presence of hydroxyapatite biomaterials from the three selected protocols; HAPS P1, HAPS P2, HAPS P3, human allograft, bovine xenograft, in the absence of biomaterial, and without differentiation. The results after 21 days can be seen in FIG. 21, where arrows indicate the calcium deposits present in the different cultures. These deposits, observed with darker staining, are a clear indication of mineralization produced by osteoblastic cells, specialized in bone matrix production.

The cell differentiation assay allowed determining that the three production protocols of HAPS induce the differentiation of mesenchymal cells into osteoblasts, confirmed by the presence of calcium deposits. Mineralization was notable for HAPS P1, P2, and, to a lesser extent, for P3, being similar to that produced by bovine xenograft and significantly higher than that of human allograft and cells differentiated in the absence of biomaterial.

These results demonstrate that the biomaterials generated with the three protocols are osteoinductive and that there was differentiation of mesenchymal cells into osteoblasts in their presence.

### Example 11—Grinding Time

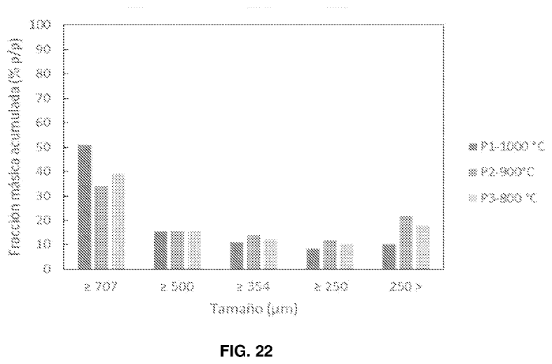

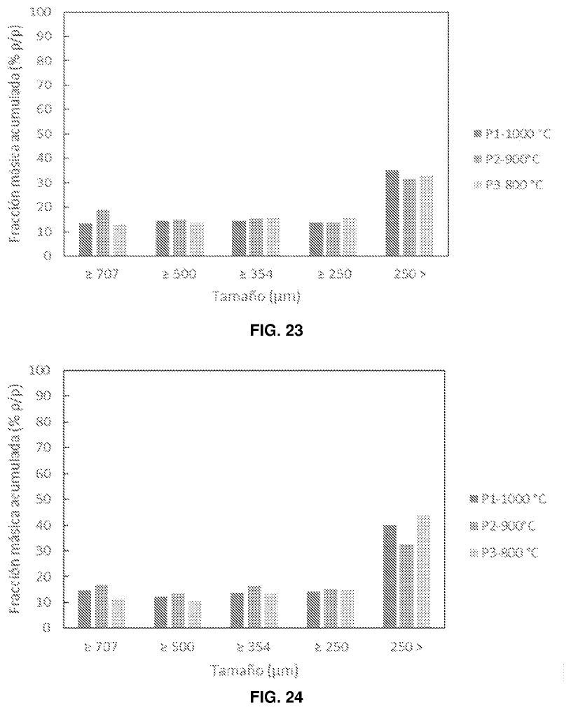

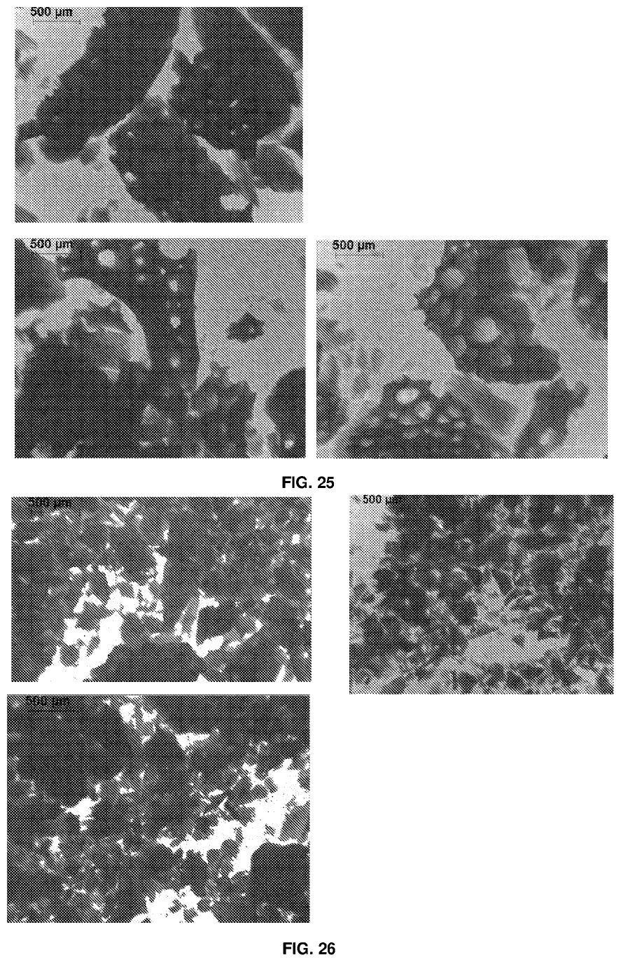

Particle sizes obtained from each of the three selected protocols using 2, 6, and 10 seconds in the grinding stage were measured. The results of the size distribution can be observed in FIGS. 22, 23, and 24. Commercially available allografts and xenografts have a particle size distribution ranging from 250 to 1000 μm. Based on this, the size distribution that most closely resembles this profile is obtained through 2-second grinding. Then, the obtained biomaterials with grinding times of 2 and 10 seconds were observed by polarized light microscopy to measure their porosity. These results can be seen in FIGS. 25 and 26.

These results led to the conclusion that a shorter grinding time generally results in larger particle size, maintaining the porosity of the biomaterial, while longer grinding time leads to smaller particle sizes and loss of biomaterial porosity. Therefore, based on the above results, grinding for 2 seconds provides a better microstructure, making it the selected condition for producing hydroxyapatite biomaterials from the selected protocols.

### Example 12—Combination of Hydroxyapatite Biomaterials with Leukocyte and Platelet-Rich Fibrin (L-PRF) and Clinical Context Evaluation

Studies were conducted on the behavior of hydroxyapatite biomaterial when combined with leukocyte and platelet-rich fibrin (L-PRF), obtaining the results shown in Table 9, where each attribute was assigned a maximum value of 5 points evaluated according to its qualitative nature.

| Attribute | P1 | P2 | P3 |
|---|---|---|---|
| Surgeon Handling | Easy to take to | Not difficult to | Easy to shape as |
| | surgical site, can be | handle. Easy to | desired in |
| | shaped as desired. | shape as desired in | small portions, easy |
| | | small portions, easy | handling. |
| | | handling. | |
| Hydrophobicity | The biomaterial | The biomaterial | The biomaterial |
| | absorbs liquid. | absorbs liquid. | absorbs liquid. |
| Aggregation | Agglutinates within | Fragile material, | Material agglutinates |
| | 5 minutes. | agglutinates in small | but separates easily. |
| | | portions. | |
| Consistency | Firm, withstands | Soft material. | Firm material that |
| | pressure. | | withstands little |
| | | | pressure. |
| Total Score Obtained | 20 | 15 | 17 |

The biomaterial obtained using protocol 1 (higher calcination temperature) received a perfect score in the combination with L-PRF evaluation. With this hydroxyapatite biomaterial, no loss of material occurred during handling. Biomaterials from protocols P1 and P2 showed excess L-PRF absorption but not completely.

Additionally, P1 and P2 did not exhibit complete aggregation, losing biomaterial particles. Despite this, the biomaterials from P2 and P3 obtained acceptable scores in the evaluation.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, gomboc, circular/circle, rectangular/rectangle, triangular/triangle, cylindrical/cylinder, elliptical/ellipse, (n) polygonal/(n) polygon, etc.; angular orientation-such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed is:

1. An aquatic-derived biomaterial, comprising:
a first composition present in the biomaterial in an amount greater than or equal to 10 wt % and less than or equal to 100 wt % versus the total biomaterial weight;
a second composition present in the biomaterial in an amount greater than or equal to 0.1 wt % and less than or equal to 90 wt % versus the total biomaterial weight;
the first composition comprising:
greater than or equal to 50 wt % and less than or equal to 90 wt % an aquatic-derived hydroxyapatite or derivative thereof, optionally substituted;
greater than or equal to 5 wt % and less than or equal to 40 wt % amorphous biomaterial, the amorphous biomaterial comprising:
greater than or equal to 10 wt % and less than 20 wt % β-tricalcium phosphate versus the total weight of the first composition,
greater than or equal to 0.1 wt % and less than or equal to 5 wt % calcium oxide,
greater than or equal to 0.1 wt % and less than or equal to 20 wt % magnesium oxide;
and
the second composition comprising one or more of collagen and/or derivatives thereof, synthetic collagen and/or derivatives thereof, gelatin and/or derivatives thereof, fibrin and/or derivatives thereof, fibrin and/or derivatives thereof.

2. An aquatic-derived biomaterial as in claim 1, wherein the aquatic-derived biomaterial is substantially free of synthetic polymers.

3. An aquatic-derived biomaterial as in any preceding claim, wherein the second composition comprises a porogenic additive and/or a bone morphogenetic protein.

4. An aquatic-derived biomaterial as in any preceding claim, wherein the collagen, actin, and/or gelatin, or derivatives thereof, is each aquatically-derived.

5. An aquatic-derived biomaterial as in any preceding claim, wherein platelet rich-fibrin is present in the biomaterial in an amount greater than or equal to 0.1 wt % and less than or equal to 5 wt % versus the total biomaterial weight.

6. An aquatic-derived biomaterial as in any preceding claim, wherein the ratio of hydroxyapatite to the second composition is greater than or equal to 70:30 and less than or equal to 90:10.

7. A method for producing an aquatic-derived biomaterial, the method comprising:
removing non-bone material comprising incubating an aquatic-derived bone for greater than or equal to 20 minutes to less than or equal to 4 hours at a temperature of greater than or equal to 700 °C and less than or equal to 120 °C thereby removing non-bone material;
deproteinizing the aquatic-derived bone comprising incubating the aquatic-derived bone in sodium hydroxide at a temperature of greater than or equal to 15 °C and less than or equal to 25 °C for greater than or equal to 30 minutes to less than or equal to 4 hours, thereby deproteinizing the bone;
delipidating the aquatic-derived bone comprising incubating the aquatic-derived bone in acetone at a temperature of greater than or equal to 15 °C and less than or equal to 30 °C for greater than or equal to 4 hours to less than or equal to 8 hours, thereby delipidating the bone;
drying the aquatic-derived bone at a temperature of greater than or equal to 50 °C and less than or equal to 70 °C for greater than or equal to 6 hours and less than or equal to 48 hours;
grinding the dried bone;
heating the ground bone to a temperature of greater than or equal to 800 °C and less than or equal to 1200 °C for greater than or equal to 1 hour and less than or equal to 3 hours thereby producing a aquatic-derived biomaterial; and
adding a second composition comprising one or more of collagen and derivatives thereof, actin and derivatives thereof, gelatin and derivatives thereof, porogenic additives, and bone morphogenetic proteins to the aquatic-derived biomaterial.

8. An article or method as in any preceding claim, wherein the aquatic-derived biomaterial is derived from one or more of fish, marine mammals, crustaceans, marine reptiles, and marine organisms.

9. An article or method as in any preceding claim, wherein the fish is selected from the group consisting of salmon, tuna, anchovy, angelfish, catfish, cod, sole, pike, char, herring, shark, kill fish, blowfish, bluefish, trout, eel, stingray, puffer fish, flounder, suckerfish, sturgeon, tigerfish, and grouper.

10. An article or method as in any preceding claim, wherein the aquatic-derived biomaterial is free of non-aquatic derived hydroxyapatite.

11. An article or method as in any preceding claim, wherein the first composition comprises an aquatic-derived hydroxyapatite and/or derivatives thereof.

12. An article or method as in any preceding claim, wherein at least a portion of the first composition is derived from salmon bone.

13. An article or method as in any preceding claim, wherein the aquatic-derived hydroxyapatite is present in the first composition in an amount greater than or equal to 54 wt % and less than or equal to 65 wt %.

14. An article or method as in any preceding claim, wherein a ratio of hydroxyapatite or derivative thereof to collagen is less than or equal to 95:5.

15. An article or method as in any preceding claim, wherein the first composition further comprises β-rhenanite, if present, in an amount greater than or equal to 0.1 wt % and less than or equal to 20 wt % versus the total weight of the first composition.

16. An article or method as in any preceding claim, wherein the first composition has density of greater than or equal to 2 g/cm³ and less than or equal to 4.2 g/cm³ as measured using gas pycnometry.

17. An article or method as in any preceding claim, wherein the first composition is thermally stable at a temperature greater than or equal to 0 °C and less than or equal to 1000 °C as measured using thermogravimetric analysis.

18. An article or method as in any preceding claim, wherein the first composition has a calcium to phosphorous ratio (Ca/P) of greater than or equal to 1.5 and less than or equal to 1.8 as measured using scanning electron microscopy coupled with energy dispersive x-ray spectroscopy (SEM-EDX).

19. An article or method as in any preceding claim, wherein the first composition has a maximum compression force of greater than or equal to 250 N and less than or equal to 600 N as measured using a TA.XT plus texture analyzer.

20. An article or method as in any preceding claim, wherein the second composition comprises one or more of collagen Type I, collagen Type II, collagen Type III, collagen Type IV and/or derivatives thereof.

21. An article or method as in any preceding claim, wherein the second composition comprises bovine derived collagen.

22. An article or method as in any preceding claim, wherein the porogenic additive comprises an oversaturated salt solution comprising an alkali, alkali earth material and halide, inorganic partially neutralized acid, and/or a neutralized organic acid.

23. An article or method as in any preceding claim, wherein the first incubation step is performed in air or an aqueous solution.

24. An article or method as in any preceding claim, wherein the deproteinizing step comprises incubating the aquatic-derived bone in an alkaline solution.

25. An article or method as in any preceding claim, wherein delipidating the aquatic-derived bone comprises incubating the aquatic-derived bone in a dilipidation solvent.

26. An article or method as in any preceding claim, wherein the aquatic-derived bone is ground to form particles of the aquatic-derived bone.

27. An article or method as in any preceding claim, wherein grinding is performed for greater than or equal to 1 second and less than or equal to 12 seconds.

28. An article or method as in any preceding claim, wherein grinding the aquatic-derived bone results in a plurality of particles.

29. An article or method as in any preceding claim, wherein at least a portion of the plurality of particles have a particle size of greater than or equal to 250 microns and less than or equal to 1000 microns.

30. An article or method as in any preceding claim, wherein the first composition and second composition are combined such that a ratio of hydroxyapatite to the second present in the aquatic-derived biomaterial is greater than or equal to 70:30 and less than or equal to 90:10.

31. A composition of a non-cytotoxic biomaterial for promoting osteogenesis comprising hydroxyapatite, characterized in that it comprises between 54 and 65% (w/w) of hydroxyapatite obtained from salmon bone, between 12 and 16% (w/w) of β-tricalcium phosphate, between 1 and 2% (w/w) of calcium oxide, between 0 and 2% (w/w) of magnesium oxide, and between 19 and 32% (w/w) of amorphous biomaterial; wherein said composition additionally includes collagen in a concentration between 10 and 90% (w/w) of the final composition.

32. The composition according to claim 31, characterized in that said biomaterial has a density between 3.1 g/cm³ and 3.4 g/cm³.

33. The composition according to claim 31, characterized in that said biomaterial is thermostable between 50 °C and 800 °C.

34. The composition according to claim 31, characterized in that said collagen is selected from the group consisting of type 1 collagen, type 2 collagen, type 3 collagen, and type 4 collagen.

35. The composition according to any one of claims 31 to 34, characterized in that it additionally comprises platelet-rich fibrin.

36. The composition according to any one of claims 31 to 35, characterized in that it is in solid, semi-solid, or liquid form.

37. Use of the composition according to any one of claims 31 to 36, characterized in that it is for the manufacture of a product useful for promoting osteogenesis.
